(19) United States
(12) Patent Application Publication
(10) Pub. No.: US 2011/0093983 A1
(43) Pub. Date: Apr. 21, 2011

(54) ALGAL GLYCEROL-3 PHOSPHATE ACYLTRANSFERASE

(75) Inventors: Jitao Zou, Saskatoon (CA); Zhifu Zheng, Zionsville, IN (US); Jingyu Xu, Saskatoon (CA)

(73) Assignee: National Research Council of Canada, Ontario (CA)

(21) Appl. No.: 12/736,170

(22) PCT Filed: Mar. 26, 2009
(86) PCT No.: PCT/US09/01912

§ 371 Date: Dec. 1, 2010

Related U.S. Application Data
(60) Provisional application No. 61/072,083, filed on Mar. 26, 2008.

(51) Int. Cl. A01H 1/00; C12P 7/64; C12N 9/10; C12N 1/19; C12N 1/13; C12N 15/63; C07H 21/00; C11B 1/00; C11B 7/00; A01H 5/00; A01H 5/10

(52) U.S. Cl. 800/281; 435/134; 435/193; 435/254.2; 435/257.2; 435/320.1; 536/23.2; 554/9; 554/175; 800/295; 800/298; 800/306; 800/320

(57) ABSTRACT

The present disclosure relates to the isolation of, purification of, characterization of, and uses for a glycerol-3-phosphate acyltransferase (TpGPAT1), and genes encoding TpGPAT1, from algae.

### CROSS-REFERENCE TO RELATED APPLICATION

This application claims the benefit under 35 U.S.C. §119(e) of U.S. Provisional Patent Application Ser. No. 61/072,083, filed Mar. 26, 2008, for “ALGAL GLYCEROL-3-PHOSPHATE ACYLTRANSFERASE,” the contents of the entirety of which are incorporated herein by this reference.

### TECHNICAL FIELD

The invention relates generally to biotechnology and, more particularly, to genes useful for the genetic manipulation of plant characteristics. In certain embodiments, the disclosure relates to isolated and/or purified polypeptides and nucleic acids encoding glycerol-3-phosphate acyltransferase (TpGPAT1) and methods of their use.

### BACKGROUND

The majority of fatty acids synthesized in eukaryotic diatoms are incorporated into either membrane glycerolipids or the neutral glycerolipid triacylglycerols (TAGS). The initial step of glycerolipid biosynthesis in diatoms is the fatty acid acylation of glycerol-3-phosphate (G-3-P) at the sn-1 position by G-3-P acyltransferase (GPAT) to form lysophosphatidic acid (LPA). LPA acyltransferase (LPAT) then catalyzes the acylation of LPA at the sn-2 position to generate phosphatidic acid (PA), which serves as a general precursor for all glycerophospholipids, including TAG. TAG synthesis involves further conversion of PA into diacylglycerol (DAG) by PA phosphatase, and subsequent acylation of DAG by either fatty acyl-CoA-dependent or phospholipid-dependent DAG acyltransferases.

### DISCLOSURE OF THE INVENTION

Eukaryotic diatoms have a unique fatty acid profile, distinctive in their high levels of 16:0, 16:1 ω 7 and 20:5 ω 3 and a low content of C18 fatty acids. The fatty acid composition of the marine diatom Thalassiosira pseudonana is typical of most diatoms, with a predominance of 16:0, 16:1 ω 7 and 20:5 w 3. However, small amounts of 18:4 w 3 and 20:6 ω 3, not usually found in diatoms, are also present.

Identified is the glycerol-3-phosphate acyltransferase (GPAT) from T. pseudonana. T. pseudonana shows a unique fatty acid profile. GPAT's role in determining such a profile has remained unknown however. GPAT catalyzes a rate-limiting and committed step of glycerolipid synthesis, thereby serving as a potential target for genetic engineering of glycerolipid biosynthesis.

Heretofore, only a small number of microsomal GPAT, which are thought to mediate oil synthesis as well as membrane lipid synthesis in eukaryotes, have been reported. These microsomal GPAT were identified from the unicellular eukaryotes Saccharomyces cerevisiae (Zheng and Zou, 2001; Zaremberg and McMaster 2002), Plasmodium falciparum (Santiago et al., 2004) and Leishmania major (Zufferey and Marnoun, 2005) and the higher eukaryotes Arabidopsis (Zheng et al., 2003) and human and mouse (Cao et al., 2006). The sequences of these enzymes are highly divergent except for the conserved acyltransferase domains.

A gene encoding a membrane-bound glycerol-3-phosphate acyltransferase, designated TpGPAT, from the marine diatom T. pseudonana has been isolated. Heterologous expression of TpGPAT in a yeast GPAT mutant (gat1) conferred a seven-fold increase in GPAT activity. Enzyme property assessment using microsomal proteins indicated that TpGPAT was highly specific for 16:0. Accordingly, expression of TpGPAT in gat1 resulted in approximately a 12% and an 18% increase of 16:0 in phospholipids and triacylglycerols, respectively. The unsaturated fatty acids, 16:1 and 18:1, on the other hand, were reduced by 15% and 21% respectively in these two lipid species. The results indicate that TpGPAT exerts a key role in determining the fatty acid composition in glycerolipids and can be used to alter fatty acid composition and/or increase oil synthesis in oil-producing organisms.

Described is a GPAT that shows highly specific preference for palmitate (16:0) and its expression in yeast dramatically increases 16:0 contents in both phospholipids and triacylglycerols. Thus, TpGPAT can be used to produce 16:0-rich oil in the oil-producing organisms such as oilseeds (e.g., Brassica, sunflower, flax, soybean, etc) through overexpression.

Generally, the fatty acid composition of glycerolipids is dictated by at least three factors: (i) the size of individual fatty acyl pools, (ii) the relative activity and specificity of fatty acyltransferases, and (iii) the relative activity and specificity of enzymes responsible for the deacylation-reacylation process. Herein, we elucidate the role of GPAT in determining the glycerolipid fatty acid composition in T. pseudonana. GPAT catalyzes the initial and committed step of glycerolipid synthesis, acylating glycerol-3-phosphate to form lysophosphatidic acid (LPA), which is further acylated by LPA acyltransferase to yield phosphatidic acid (PA) as a general precursor for all glycerophospholipids. Herein, we identify TpGPAT (or “TpGPAT1”), encoding a membrane-bound GPAT, from T. pseudonana. GPAT prefers 16:0-CoA as acyl donor and mediates the synthesis of glycerolipid molecules enriched with 16:0 fatty acid.

Glycerolipids of the marine diatom T. pseudonana have predominantly 16:0, 16:1 ω 7 and 20:5 ω 3 fatty acids. We studied a membrane bound glycerol-3-phosphate acyltransferase (GPAT), designated TpGPAT, in this algal species. The primary sequence of TpGPAT is composed of 674 amino acids, which shows much higher similarity to the GPAT sequences from the unicellular eukaryotes Saccharomyces cerevisiae, Leishmania major, and Plasmodium falciparum than to those from higher plants and mammals.

A transgenic plant containing a nucleic acid construct is also disclosed. A method of transforming a cell or a plant is described; the method comprising introducing the isolated, purified or recombinant nucleic acid into the cell or plant. A process for producing a genetically transformed plant seed comprises introducing the nucleic acid into the plant seed. In some embodiments, these methods may be used for modifying plants to change their seed oil content.

Expression of TpGPAT in, for example, canola, soybean, and other oilseeds is expected to produce high-palmitate oils. Such oils can be used for the production of margarine and as oleochemical, soap, and animal feed raw material. Whereas oils with high contents of long-chain or very long-chain polyunsaturated fatty acids are desirable for many purposes including human nutrition, oils with highly saturated 16-carbon-chain length fatty acids can provide the starting materials for many industrial applications.

Also described is a process of producing biodiesel from algal cells, wherein the improvement comprises using, as an algal cell in the process, the algal cell transformed as described herein to overexpress TpGPAT.

### BRIEF DESCRIPTION OF THE FIGURES

FIG. 1A is an alignment of the four conserved acyltransferase domains of TpGPAT (SEQ ID NO:2) with glycerol-3-phosphate acyltransferases (GPAT) and dihydroxyacetone-phosphate acyltransferase (DHAPAT) from other species. PfGAT from P. falciparum (accession no. XP_{—}001350533; SEQ ID NO:12); LmGAT from L. major (accession no. XP_{—}001687304; SEQ ID NO:13); Gat1p (SEQ ID NO:14) and Gat2p (SEQ ID NO:15) from S. cerevisiae (accession no. AJ311354 and AJ314608, respectively); hGPAT1 (SEQ ID NO:16), hGPAT2 (SEQ ID NO:17) and hGPAT3 (SEQ ID NO:18) from H. sapiens (accession no. NP_{—}065969; AAH68596 and NP_{—}116106, respectively); mGPAT1 (SEQ ID NO:19), mGPAT2 (SEQ ID NO:20) and mGPAT3 (SEQ ID NO:21) from M. musculus (accession no. NP_{—}032175, BAF03614 and NP_{—}766303, respectively); P1sB (SEQ ID NO:22) from E. coli (accession no. NP_{—}756863); AtGPAT1 (SEQ ID NO:23) and AtGPAT6 (SEQ ID NO:24) from A. thaliana (accession no. At1g06520 and At2g38110, respectively); hDHAPAT (SEQ ID NO:25) from H. sapiens (accession no. BAD96493); mDHAPAT (SEQ ID NO:26) from M. musculus (accession no. NP_{—}034452); and LmDAT (SEQ ID NO:27) from L. major (accession no. XP_{—}001686202). Identical amino acid residues are highlighted in black, and conserved residues are shaded. The four conserved acyltransferase motifs are donated with motifs I, II, III and IV, respectively.

FIG. 1B is a phylogenic tree of TpGPAT1 and acyltransferases from other species. The partial amino acid sequences encompassing the 4 acyltransferase motifs were aligned using the Clustal W method of Lasergene analysis software (DNAStar, Madison, Wis.).

FIG. 1C is the predicted topology of TpGPAT1 using the TMHMM algorithm indicating the presence of five transmembrane domains. AT1, AT2, AT3 and AT4 represent the four conserved acyltransferase motifs.

FIG. 2 depicts GPAT activity of TpGPAT1 expressed in yeast gat1 mutant. The microsomal membrane fractions prepared from lysates of the induced yeast cells harboring TpGPAT1 or empty vector pYES2.1 were assayed for GPAT activity with 400 μM [¹⁴C]glycerol 3-phosphate (2.5 nCi/nmol), 45 μM palmitoyl-CoA, 75 mM Tris-HCl, pH 7.5, 1 mM DTT, and 2 mM MgCl₂ for 10 min at room temperature. After extraction of the phospholipid products, the radioactivity was measured by scintillation count.

FIG. 3 depicts substrate specificity of TpGPAT1. The microsomal membrane fractions prepared from lysates of the induced yeast cells harboring TpGPAT1 or empty vector pYES2.1 were assayed for GPAT activity with 400 μM [¹⁴C]glycerol 3-phosphate (2.5 nCi/nmol) and different acyl-CoAs as acyl donor. After extraction of the phospholipid products, the radioactivity was measured by scintillation count.

FIG. 4 graphically depicts the results of the lipidomic analysis (fatty acid composition) of the lipids from yeast gat1 mutants transformed with either TpGPAT1 or GAT1. Yeast cells expressing TpGPAT or yeast GAT1 were fed with EPA (20:5) or DHA (22:6) upon induction of the genes. Total lipids from the yeast cells were extracted and subjected to lipidomic analysis using a tandem mass spectrometer. The results are presented as the molar ratio of EPA- or DHA-containing phospholipids (PC, PE, PS, PI) in the total phospholipids.

FIG. 5 graphically depicts the results of the analysis of the fatty acid 16:0 content of T2 seeds from TpGPAT transgenic Arabidopsis. Fatty acid analysis was performed on TpGPAT transformed Arabidopsis seeds. Fatty acid composition (as molar percentage) was determined in the seed oil extracted from 200 T2 seeds of 13 TpGPA:pSE transformed Arabidopsis lines (GW4-GW17). pSE129A empty plasmid transformed wild-type Arabidopsis was used as a control.

FIG. 6 graphically depicts the results of the analysis of the fatty acid 16:0 content of T1 seeds from TpGPAT transgenic Brassica napus. Fatty acid analysis was performed on TpGPAT transformed B. napus seeds. Fatty acid composition (as molar percentage) was determined in the seed oil extracted from T1 seeds of five independent TpGPAT:pSE transformed Brassica napus events (GPAT1, 3, 4, 7 and 12). Wild-type B. napus was used as a control.

FIG. 7 graphically depicts the total oil content of TpGPAT transformed Arabidopsis seeds. Oil contents (as percentage of dry weight) were determined in 200 T2 seeds of the 13 TpGPA:pSE transformed Arabidopsis lines (GW4-12; GW14-17). Wild-type Arabidopsis was used as a control (Con).

### DETAILED DESCRIPTION

```
The nucleotide sequence of T. pseudonana GPAT
  (SEQ ID NO: 1)
ATGGGCGTTGAAAAGAAGGGCACAATGATGTCCGAGTTGGACTATACGA
AGGCACAACTCGCCTTCTTCTACATCGTCGTCCTTCTATCACTCGATAT
GCTCAACCCAGTCAAGATCTTTTTACACGTCTTTCCTGCAATTAAGTCA
TGGCACATCGCGACATTTGCAATTGCCTGCATGTCATACATCTTCATCG
TGAACTTGAGGGAACTGCTATACTTCGCTACCAAGGTCTTCTTCCATTC
AATCCTATCAATCTTTTTCAACGACGTGACCGTGGTTGGCAGAGAGAAT
ATCCCGAGCCATGGCCCTGTTATCTTTACCTCCAACCACGCTAATCAGT
TTATGGATGGGTTGATGATTATGTGTACTTGCCAAAGGACGATCTCGTA
TCTTGTAGCAGACAAGTCTTGGAATAGACCAATCATTGGGCATCTGGCT
TGGATGATGGGGGGAGTGCCAGTCAAACGTGCACAAGATAGTGCCTGTA
AAGGAACTGGAAAAATCAGCATTGACTTGAACGCTCTCGCGGGATCGGA
TGCAGTCATCAATGTCGTTGGAAAGGGAACATCGTTCACGTCTCAGATA
AAAGCCGGGGATAAGATTCGCCTACCAAACAATGCAATCGGCATCAAAG
TTGAATCTATCGAAAGTGATGAATCAATGTCGCTCAAAGTGGAAGATGG
TGTGGCTGAAGTATTATCATCCCATCCATTTCCTGAGTACGTCACATAC
GATATTCTGCCTCGAATTGATCAGAAGGACGTCTACCAAAATGTACTGG
AGAAACTAGCATCAGGCGGGACGATTGGAATCTTTCCAGAGGGTGGCTC
CCACGATAGGACTGACTTGCTCCCATTGAAAGTTGGTGTGGCACTCATT
GCATACTCGGAACTTGAAAAGGATGGAATCAACGTGCCGATTGTCCCAG
TTGGATTGAACTACTTTCGGGCTCATCGCTTCCGTGGCAAAGCAACTGT
TGAGTTTGGTTCTCCAACTTATATTGAACCATCGACACTTGCAGACTAC
AAAAAGGGAGGTGCCGATAAGCGACGCGTTTGCAATGATCTTTTGGCTC
GTATTGAGAACAGTATGAGATCTGTCATTGTGTCGGTGCCCGACTTTGA
AACACTTCAGACCATCCATGCTGCGAGGAGGCTATACAGACAAGATGGC
AGAAACGAAACTGCTGAACAAAGGCAGGACATGGGCAGGCGATTTGCAG
AGGGGTACAAACGCGTTCTTCTCCAATTAGGAGGAGAGCCGCCAGAGGA
GTGGCTCAGTCTGCAGTCGAGGATATTGGCGTACCAAAAGGAATTGAAC
GAGCTCGGTATCAGGGACTATCAGGTTGTTGGTCTTGATCACGAGGAGG
TGGAACTCGGTTCAGAGTCGCAAGGTCATTCCAAAGCAGATACTGTTCT
TCACAGGATGAATGTGTTTGGACACATCGTTCATCTGTTTGTCATTGCG
GTCTTGGCAGCACTGCCAGCTATGTTGTTAAACCTTCCAGTTGGGTTGG
CATCACGAATCTACTCCAATCGTAGGCGAAAGGTTGCATTGGCAGCATC
AAAAGTGAAGGTGAAGGGATATGATGTGATGCTCTCAGAACGTGTACTG
GCATGTATCGTTCTCGTTCCTTCTCTGTGGGTAGTGTATGGATTGCTTC
TCTCCCTGTTCACCTCTCTCGACGGACCGTCACTTGCTGTATGCTTCAC
CTGCTTTCCTTTGTTCTCATATTGGAGTATTATGGCTACGGAATCAGGA
ATGGTTGATATCAAAGATTTGAGGCCGTACGTTATGAGAATGATTCCAT
CAGCACGACGCAGGTTAGCAGCATTGCCAGCAACGAGGAAGGCTCTCCG
GTCGGATCTTAGAGCAATGATTAAGAAGATTGGCCCCAGCTTGGGTGAT
ATTTACTACGAAAAGGACTTGAACTGGCAGAAGATTCAAATGGAGACGA
AGAGGATGTCAATGGAGGAGTTGGATCCAGCTCAAAAAGACGAAGCAGC
GAAGAAGGAAGAGTAA
The amino acid sequence of T. pseudonana GPAT
  (SEQ ID NO: 2)
MGVEKKGTMMSELDYTKAQLAFFYIVVLLSLDMLNPVKIFLHVFPAIKS
WHIATFAIACMSYIFIVNLRELLYFATKVFFHSILSIFFNDVTVVGREN
IPSHGPVIFTSNHANQFMDGLMIMCTCQRTISYLVADKSWNRPIIGHLA
WMMGGVPVKRAQDSACKGTGKISIDLNALAGSDAVINVVGKGTSFTSQI
KAGDKIRLPNNAIGIKVESIESDESMSLKVEDGVAEVLSSHPFPEYVTY
DILPRIDQKDVYQNVLEKLASGGTIGIFPEGGSHDRTDLLPLKVGVALI
AYSELEKDGINVPIVPVGLNYFRAHRFRGKATVEFGSPTYIEPSTLADY
KKGGADKRRVCNDLLARIENSMRSVIVSVPDFETLQTIHAARRLYRQDG
RNETAEQRQDMGRRFAEGYKRVLLQLGGEPPEEWLSLQSRILAYQKELN
ELGIRDYQVVGLDHEEVELGSESQGHSKADTVLHRMNVFGHIVHLFVIA
VLAALPAMLLNLPVGLASRIYSNRRRKVALAASKVKVKGYDVMLSERVL
ACIVLVPSLWVVYGLLLSLFTSLDGPSLAVCFTCFPLFSYWSIMATESG
MVDIKDLRPYVMRMIPSARRRLAALPATRKALRSDLRAMIKKIGPSLGD
IYYEKDLNWQKIQMETKRMSMEELDPAQKDEAAKKEE
```

Some of the manipulations that are possible using the TPGPAT1 gene or a part thereof, include, but are not limited to, the following: seeds or plants with increased or decreased oil content; seeds or plants containing oils with an enhanced polyunsaturated fatty acid content, and plants exhibiting an enhanced or altered capacity to accumulate various fatty acids.

In order to facilitate review of the various embodiments of the disclosure, the following explanations of specific terms are provided:

Degree or percentage of sequence homology: The term “degree or percentage of sequence homology” refers to degree or percentage of sequence identity between two sequences after optimal alignment. Percentage of sequence identity (or degree of identity) is determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity.

Homologous isolated and/or purified sequence: “Homologous isolated and/or purified sequence” is understood to mean an isolated and/or purified sequence having a percentage identity with the bases of a nucleotide sequence, or the amino acids of a polypeptide sequence, of at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, or 99.7%. This percentage is purely statistical, and it is possible to distribute the differences between the two nucleotide sequences at random and over the whole of their length. Sequence identity can be determined, for example, by computer programs designed to perform single and multiple sequence alignments. It will be appreciated that this disclosure embraces the degeneracy of codon usage as would be understood by one of ordinary skill in the art. Furthermore, it will be understood by one skilled in the art that conservative substitutions may be made in the amino acid sequence of a polypeptide without disrupting the structure or function of the polypeptide. Conservative substitutions are accomplished by the skilled artisan by substituting amino acids with similar hydrophobicity, polarity, and R-chain length for one another. Additionally, by comparing aligned sequences of homologous proteins from different species, conservative substitutions may be identified by locating amino acid residues that have been mutated between species without altering the basic functions of the encoded proteins.

Isolated: As will be appreciated by one of skill in the art, “isolated” refers to polypeptides that have been “isolated” from their native environment.

Nucleotide, polynucleotide, or nucleic acid sequence: “Nucleotide, polynucleotide, or nucleic acid sequence” will be understood as meaning both a double-stranded and single-stranded DNA in the monomeric and dimeric (so-called in tandem) forms and the transcription products of the DNAs.

Sequence identity: Two amino-acids or nucleotide sequences are “identical” if the sequence of amino-acids or nucleotide residues in the two sequences is the same when aligned for maximum correspondence as described below. Sequence comparisons between two (or more) peptides or polynucleotides are typically performed by comparing sequences of two optimally aligned sequences over a segment or “comparison window” to identify and compare local regions of sequence similarity. Optimal alignment of sequences for comparison may be conducted by the local homology algorithm of Smith and Waterman, Ad. App. Math 2: 482 (1981), by the homology alignment algorithm of Neddleman and Wunsch, J. Mol. Biol. 48: 443 (1970), by the search for similarity method of Pearson and Lipman, Proc. Natl. Acad. Sci. (U.S.A.) 85: 2444 (1988), by computerized implementation of these algorithms (GAP, BESTFIT, FASTA, and TFASTA in the Wisconsin Genetics Software Package, Genetics Computer Group (GCG), 575 Science Dr., Madison, Wis.), or by visual inspection. The definition of sequence identity given herein is the definition that would be used by one of skill in the art. The definition by itself does not need the help of any algorithm, the algorithms being helpful only to achieve the optimal alignments of sequences, rather than the calculation of sequence identity. From the definition given herein, it follows that there is a well defined and only one value for the sequence identity between two compared sequences which value corresponds to the value obtained for the best or optimal alignment.

In the BLAST N or BLAST P “BLAST 2 sequence,” software which is available in the web site http://worldwideweb.ncbi.nlm.nih.gov/gorf/b12.html, and habitually used by the inventors and in general by the skilled man for comparing and determining the identity between two sequences, gap cost which depends on the sequence length to be compared is directly selected by the software.

Stringent hybridization: Hybridization under conditions of stringency with a nucleotide sequence is understood as meaning hybridization under conditions of temperature and ionic strength chosen in such a way that they allow the maintenance of the hybridization between two fragments of complementary DNA. Homologs of the TPGPAT1 genes described herein obtained from other organisms, for example plants, may be obtained by screening appropriate libraries that include the homologs, wherein the screening is performed with the nucleotide sequence of the specific TpGPAT1 genes disclosed herein, or portions or probes thereof, or identified by sequence homology search using sequence alignment search programs such as BLAST, FASTA.

Proteins that are homologous to full-length T. pseudonana TPGPAT1 can be found by searching protein databases, such as the NCBI protein database, with search engines, such as BLAST. They may also be identified by rational design. The process of rational design may comprise identifying conservative amino acid substitutions within the desired polypeptide sequence length, and making those substitutions in the encoded protein.

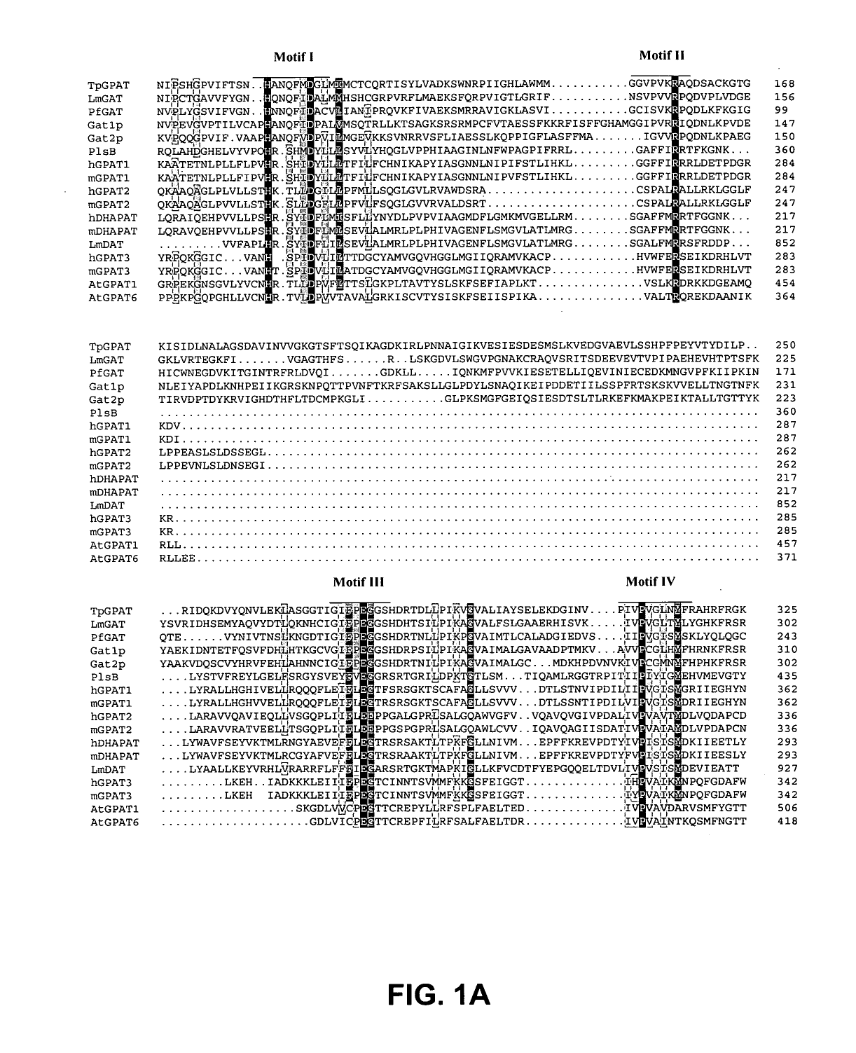

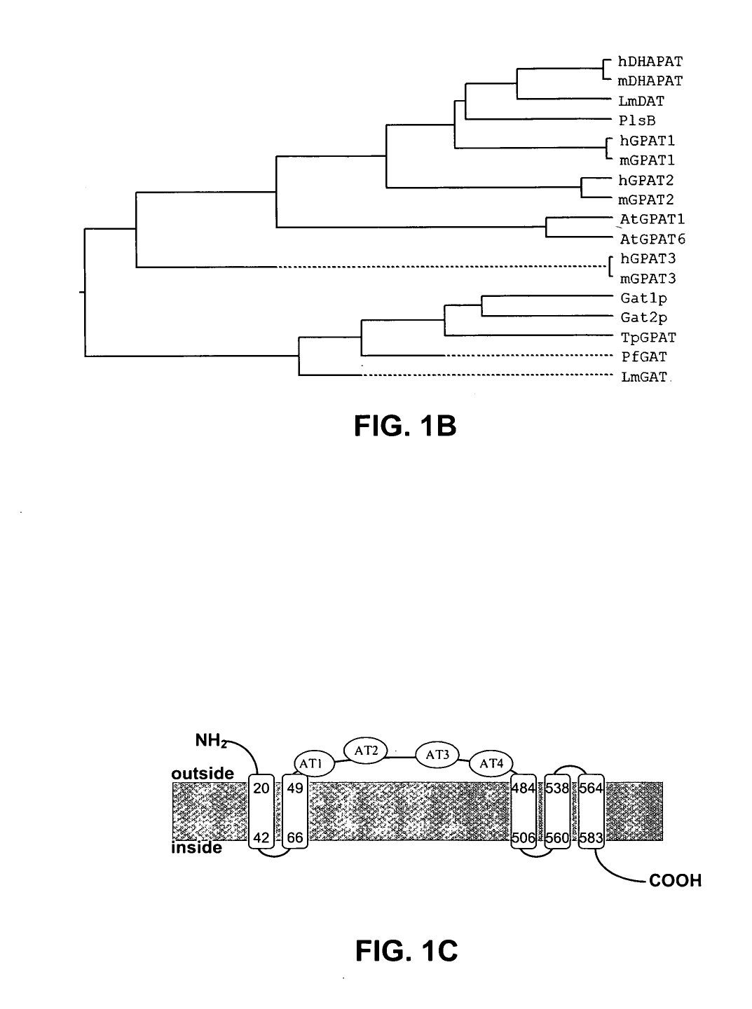

Searching the NCBI protein database with the full-length amino acid sequence of T. pseudonana TpGPAT1 (BLASTP) reveals polypeptides with significant sequence homology to TpTPGPAT1, several of which are shown aligned with TpTPGPAT1 in FIG. 1A. The conserved diacylglycerol transferase domain is described within NCBI's conserved domain database. (WorldWideWeb.ncbi.nlm.nih.gov/Structure/cdd/cdd.shtml). Polypeptide sequences that are homologous to this conserved domain impart the type 2 diacylglycerol activity of TpTPGPAT1 to proteins wherein it is contained.

It is understood by those of skill in the art that polypeptides with homologous sequences may be designed to exhibit the same structure and function as their homologs. The skilled artisan is, for example, able to design homologous polypeptides to those specifically described in the Examples of this disclosure and by the sequence alignment of FIG. 1A. Such homologous polypeptides may be those that contain conservative substitutions to polypeptides of the present disclosure, for example the polypeptides of SEQ ID NOS:3 and 4. Simple experimental assays that determine which homologous proteins exhibit substantially similar diacylglycerol transferase activity to TpTPGPAT1 are known to those skilled in the art. Such assays are not unduly time-consuming, expensive, or technically difficult. For example, conventional gas chromatography may be used to detect TAG produced by TpTPGPAT1. Several of these assays are described in the detailed examples below.

Further included are nucleic acid molecules that hybridize to the herein disclosed sequences. Hybridization conditions may be stringent in that hybridization will occur if there is at least a 90%, 95% or 97% identity with the nucleic acid molecule that encodes the disclosed TPGPAT1 molecules. The stringent conditions may include those used for known Southern hybridizations such as, for example, incubation overnight at 42° C. in a solution having 50% formamide, 5×SSC (150 mM NaCl, 15 mM trisodium citrate), 50 mM sodium phosphate (pH 7.6), 5×Denhardt's solution, 10% dextran sulfate, and 20 micrograms/milliliter denatured, sheared salmon sperm DNA, following by washing the hybridization support in 0.1×SSC at about 65° C. Other known hybridization conditions are well known and are described in Sambrook, et al., Molecular Cloning: A Laboratory Manual, Third Edition, Cold Spring Harbor, N.Y. (2001), incorporated herein in its entirety by this reference.

DNA isolation and cloning is well established. Similarly, DNA encoding an isolated enzyme may be inserted into a vector and transformed into yeast cells by conventional techniques. However, because no cloned GPAT gene has been shown to efficiently use VLCPUFAs, it has not previously been possible to address the possibility of genetic modifications of GPAT, for example to modulate GPAT1 activity to efficiently use VLCPUFA. We confirmed that TPGPAT1 is involved with TAG synthesis and utilizes VLCPUFA more efficiently than other GPATs. Therefore, genetic engineering of GPAT polypeptides that comprise desirable fatty acid profiles is now possible, in view of the present disclosure.

Nucleic acid molecules that code for TPGPAT1, for example sequences having at least 80% identity to SEQ ID NO:1 may be transformed into an organism, for example a plant. Such homologous sequences are exemplified by SEQ ID NOS:5-6. As known in the art, a number of ways exist by which genes and gene constructs can be introduced into organisms, for example plants, and a combination of transformation and tissue culture techniques have been successfully integrated into effective strategies for creating transgenic organisms, for example crop plants. These methods have been described elsewhere (Potrykus, 1991; Vasil, 1994; Walden and Wingender, 1995; Songstad, et al., 1995), and are well known to persons skilled in the art. For example, one skilled in the art will certainly be aware that, in addition to Agrobacterium-mediated transformation of Arabidopsis by vacuum infiltration (Bechtold et al., 1993) or wound inoculation (Katavic, et al., 1994), it is equally possible to transform other plant and crop species, using Agrobacterium Ti-plasmid mediated transformation (e.g., hypocotyl (DeBlock, et al., 1989) or cotyledonary petiole (Moloney, et al., 1989) wound infection), particle bombardment/biolistic methods (Sanford, et al., 1987; Nehra, et al., 1994; Becker, et al., 1994) or polyethylene glycol-assisted, protoplast transformation (Rhodes, et al., 1988; Shimamoto, et al., 1989) methods.

Many examples exist of successful modifications to plant metabolism that have been achieved by genetic engineering to transfer new genes or to alter the expression of existing genes, in plants. It is now routinely possible to introduce genes into many plant species of agronomic significance to improve crop performance (e.g., seed oil or tuber starch content/composition, meal improvement; herbicide, disease or insect resistance, heavy metal tolerance, etc.) (MacKenzie and Jain, 1997; Budziszewski, et al., 1996; Somerville, 1993; Kishore and Somerville, 1993).

As will also be apparent to persons skilled in the art, and as described elsewhere (Meyer, 1995; Dada, et al., 1997), it is possible to utilize plant promoters to direct any intended up- or down-regulation of transgene expression using constitutive promoters (e.g., those based on CaMV35S), or by using promoters which can target gene expression to particular cells, tissues (e.g., napin promoter for expression of transgenes in developing seed cotyledons), organs (e.g., roots), to a particular developmental stage, or in response to a particular external stimulus (e.g., heat shock).

Promoters for use herein may be inducible, constitutive, or tissue-specific or have various combinations of such characteristics. Useful promoters include, but are not limited to, constitutive promoters, e.g., carnation etched ring virus (CERV), cauliflower mosaic virus (CaMV) 35S promoter, or more particularly the double enhanced cauliflower mosaic virus promoter, comprising two CaMV 35S promoters in tandem (referred to as a “Double 35S” promoter).

It may be desirable to use a tissue-specific or developmentally regulated promoter instead of a constitutive promoter in certain circumstances. A tissue-specific promoter allows for overexpression in certain tissues without affecting expression in other tissues. By way of illustration, a promoter used in overexpression of enzymes in seed tissue is an ACP promoter as described in PCT International Publication WO 92/18634, published Oct. 29, 1992, the contents of which is herein incorporated by reference.

The promoter and termination regulatory regions may be functional in the host plant cell and may be heterologous (that is, not naturally occurring) or homologous (derived from the plant host species) to the plant cell and the gene. Suitable promoters which may be used are described herein.

The termination regulatory region may be derived from the 3′ region of the gene from which the promoter was obtained or from another gene. Suitable termination regions which may be used are well known in the art and include Agrobacterium tumefaciens nopaline synthase terminator (Tnos), A. tumefaciens mannopine synthase terminator (Tmas) and the CaMV 35S terminator (T35S), the pea ribulose bisphosphate carboxylase small subunit termination region (TrbcS), or the Tnos termination region. Such gene constructs may suitably be screened for activity by transformation into a host plant via Agrobacterium and screening for increased isoprenoid levels.

Suitably, the nucleotide sequences for the genes may be extracted from the GenBank® (a registered trademark of the U.S. Department of Health and Human Services) nucleotide database and searched for restriction enzymes that do not cut. These restriction sites may be added to the genes by conventional methods such as incorporating these sites in PCR primers or by sub-cloning.

A DNA construct for use herein may be comprised within a vector, most suitably an expression vector adapted for expression in an appropriate host cell, for example a plant cell. It will be appreciated that any vector which is capable of producing a cell comprising the introduced DNA sequence will be sufficient.

Suitable vectors are well known to those skilled in the art and are described in general technical references, such as Pouwels et al., Cloning Vectors. A Laboratory Manual, Elsevier, Amsterdam (1986). Particularly suitable vectors include the Ti plasmid vectors.

Transformation techniques for introducing the DNA constructs into host cells are well known in the art and include such methods as micro-injection, using polyethylene glycol, electroporation, high velocity ballistic penetration, or Agrobacterium-mediated transformation. After transformation of the plant cells or plant, those plant cells or plants into which the desired DNA has been incorporated may be selected by such methods as antibiotic resistance, herbicide resistance, tolerance to amino-acid analogues, or using phenotypic markers.

Various assays may be used to determine whether the plant cell shows an increase in gene expression, for example, Northern blotting or quantitative reverse transcriptase PCR(RT-PCR). Whole transgenic plants may be regenerated from the transformed cell by conventional methods. Such transgenic plants having improved isoprenoid levels may be propagated and self-pollinated to produce homozygous lines. Such plants produce seeds containing the genes for the introduced trait and can be grown to produce plants that will produce the selected phenotype.

Particularly preferred plants for modification according to the present disclosure include Arabidopsis thaliana, borage (Borago spp.), Canola, castor (Ricinus communis) (Ricinus spp.), cocoa bean (Theobroma cacao) (Theobroma spp.), corn (Zea mays) (Zea spp.), cotton (Gossypium spp), Crambe spp., Cuphea spp., flax (Linum spp.), Lesquerella spp. and Limnanthes spp., Linola, nasturtium (Tropaeolum spp.), Oenothera spp., olive (Olea spp.), palm (Elaeis spp.), peanut (Arachis spp.), rapeseed, safflower (Carthamus spp.), soybean (Glycine spp. and Soja spp.), sunflower (Helianthus spp.), tobacco (Nicotiana spp.), Vernonia spp., wheat (Triticum spp.), barley (Hordeum spp.), rice (Oryza spp.), oat (Avena spp.) sorghum (Sorghum spp.), rye (Secale spp.) or other members of the plant family Gramineae.

Some embodiments are used to modify the yield or composition of oilseed produced from oilseed crops. Oilseed crops are plant species that are capable of generating edible or industrially useful oils in commercially significant yields, and include many of the plant species listed herein. Such oilseed crops are well known to persons skilled in the art.

In one example, plants transformed with a nucleotide sequence that codes for a TPGPAT1 are grown. Seeds of the transgenic plants are harvested and fatty acids of the seeds are extracted. The extracted fatty acids are used for subsequent incorporation into a composition, for example a pharmaceutical composition, a nutraceutical composition, or a food composition.

In certain embodiments, other methods of enhancing or altering oil production may also be used with the plant to be transformed (e.g., incorporating, for expression in the plant, a nucleic acid sequence selected from the group comprising a nucleic acid sequence encoding a peptide having, for example, Brassica pyruvate dehydrogenase kinase activity (see, e.g., U.S. Pat. No. 7,214,859 to Marilla, et al. (May 8, 2007), U.S. Pat. No. 6,500,670 to Zou, et al. (December 2002), and U.S. Pat. No. 6,256,636 to Randall, et al. (July 2001), the contents of the entirety of each of which is incorporated herein by this reference), a nucleic acid sequence encoding a peptide having diacylglycerol acyltransferase activity (see, e.g., U.S. Pat. No. 7,015,373 and U.S. Pat. No. 6,500,670 to Zou, et al. (December 2002), the contents of the entirety of each of which is incorporated herein by this reference), and a nucleic acid sequence encoding a peptide having glycerol-3-phosphate dehydrogenase activity (see, e.g., U.S. Pat. No. 7,112,724, the contents of the entirety of which is incorporated herein by this reference, and combinations thereof).

Embodiments are susceptible to various modifications and alternative forms in addition to those specific Examples described in detail herein. Thus, embodiments are not limited to the particular forms disclosed. Rather, the scope of the disclosure encompasses all modifications, equivalents, and alternatives falling within the following appended claims.

### EXAMPLES

### Example I

### Isolation of a GPAT cDNA from T. pseudonana

The draft genome of the diatom T. pseudonana was searched using the yeast Gat1p and Gat2p sequences as query. (See, Zheng and Zou, 2001). One homologous nucleotide sequence, designated TpGPAT, was retrieved and amplified by PCR as described herein.

Plasmid from a cDNA library of T. pseudonana was used as template. A 50 μl PCR reaction containing 50 ng of plasmid DNA, 20 μM of each primer: 5′-GGTATGCTCATCTGCTACCCCCTC-3′ (SEQ ID NO:7) and 5′-TTAAGTCTCCTTCGTCTTTGGTGTAG-3′ (SEQ ID NO:8) and 1 μl of BD ADVANTAGE™ 2 Polymerase Mix (Clontech Laboratories, Inc.) was incubated for 30 cycles according to the following thermocycle program: 94° C. for 30 sec., 58° C. for 30 sec., and 72° C. for 1 min. 30 sec. The PCR product was purified and subsequently cloned into the pYES2.1/N5-His-TOPO expression vector (Invitrogen).

### Example II

### Heterologous Expression of TpGPAT in Yeast

The TpGPAT in pYES2.1 N5-His-TOP0 plasmid was transformed into yeast gat1 A (BY4742, Mat α, his3C1, leu2C0, lys2C0, ura3C0, YKR067w::kanMX4) using the method provided by the producer's manual (Invitrogen). Yeast cells transformed with pYES2.1/V5-His-TOPO plasmid only were used as a control. Transformants were selected by growth on synthetic complete medium lacking uracil (SC-ura), supplemented with 2% (w/v) glucose. The colonies were transferred into liquid SC-ura with 2% (w/v) glucose and grown at 28° C. overnight. The overnight cultures were diluted to an OD 0.4 in induction medium (SC-ura+2% Galactose+1% Raffinose), and were induced by incubating at 28° C. overnight. The yeast cells were collected and broken using glass beads. The protein concentrations in the lysates were normalized using a Biorad assay (Bradford, 1976) and then assayed for GPAT activity.

Enzyme Assays: GPAT assays were conducted at 30° C. for 10 min. in a 200-pL reaction mixture containing 40 mM Hepes, pH 7.0, 400 pM ¹⁴C-glycerol-3-phosphate (2.5 nCi/nmol), 67.5 pM palmitoyl-CoA and/or stearoyl-CoA or other fatty acyl donors, 1 mM DTT, 2 mM MgCl₂, and 2.5 mg/mL BSA unless stated otherwise. The reaction was stopped, and products were extracted as described previously. (Zheng and Zou, 2001). The formed products were subjected to scintillation counting for radioactivity and thin layer chromatography analysis as described.

### Example III

### Yeast DHA/EPA Feeding and Total Lipid Analysis

Yeast cultures were grown at 28° C. in the presence of 2% (w/v) glucose and 1% (w/v) Tergitol NP-40 (Sigma, St. Louis, Mo., US). Expression of the transgene was induced at OD600 nm 0.2 to 0.3 by supplementing galactose to 2% (w/v). At that time, the appropriate FAs were added to a final concentration of 50 or 100 μM in the presence of 1% (w/v) Tergitol NP-40 (Sigma, St. Louis, Mo., US). Yeast cells (20 ml) were harvested after a 3-day incubation, total lipids from yeast homogenates were extracted using the procedure of Schneiter (2005). Separation of neutral lipids and polar lipids was performed by running the thin layer chromatography plates (Si 250-PA, Baker, Phillipsburg, N.J.) in a developing solvent of hexane: ether: acetic acid (70:30:1, v/v) and identified by co-migration with known standards. Spots corresponding to TAG and phospholipid were scraped off the TLC plates and were transmethylated with methanolic HCl and quantified by gas chromatography as described previously. (Zheng and Zou, 2001).

### Results

### Isolation and Characterization of the TpGPAT cDNA from T. pseudonana

To identify GPAT enzyme(s) from T. pseudonana, the v3.0 draft genome of this diatom was searched for DNA sequences encoding the peptide(s) similar to yeast Gat1p and Gat2p. Zheng Z., and Zou J., supra. A homologous sequence was identified, designated TpGPAT1. A full-length cDNA clone was amplified by PCR from a cDNA library of T. pseudonana. It contains an open reading frame of 2,025 bp, which encodes a polypeptide of 674 amino acids with a calculated molecular mass of 75.2 kD. The predicted coding sequence in the T. pseudonana genome data base, with the transcript sequence being 2,334 bp, could not be amplified. Comparison of the genomic and cDNA sequences of TpGPAT revealed one intron of 102 bp near the 5-′end. The amino acid sequence of TpGPAT exhibits 24% and 23% identity to yeast Gat1p and Gatzp, respectively.

A relatively high similarity to P. falciparum GPAT (PfGPAT, 27% identity) and Leishmania major GPAT (LmGPAT, 25% identity) was registered for TpGPAT. In contrast, TpGPAT shares little homology with bacterial, mammalian, and Arabidopsis membrane-bound GPATs on the full-length scale (data not shown). A remarkable feature shared among TpGPAT, yeast Gat1p and GatZp, PfGPAT and LmGPAT is a long stretch of more than 100 amino acids between conserved acyltransferase motifs II and III (FIG. 1A). This distinguishes them from other known GPATs, LPATs and dihydroxyacetone phosphate acyltransferases (DHAPATs) that are characteristic of a much shorter spacer less than 60 amino acids) between the two motifs. Together with previous findings that PfGPAT and LmGPAT are a yeast-like GPAT (Santiago et al., 2004; Zufferey and Mamoun, 2005), the results suggest that the four unicellular eukaryotes T. pseudonana, P. falciparum, L. major, and S. cerevisiae are evolutionarily closely related (FIGS. 1A & 1B).

The alignment of multiple partial sequences revealed that TpGPAT contains all four acyltransferase motifs. Motif I of TpGPAT contains the amino acid sequence, HANQFMDGLMIT. (SEQ ID NO:28). Motif II of TpGPAT contains the amino acid sequence, VPVKRAQD. (SEQ ID NO:29). Motif III of TpGPAT contains the amino acid sequence, IGIFPEGGSHD. (SEQ ID NO:30). Motif IV of TpGPAT contains the amino acid sequence, IVPVGLNY. (SEQ ID NO:31). The histidine and aspartate residues in motif I, which are catalytically important, remain invariant among all the sequences. Nevertheless, five residues, instead of four for most of known fatty acyltransferases, are present between the conserved histidine and aspartate in TpGPAT. This feature is shared among the known GPATs from unicellular eukaryotes (FIG. 1A). In motif II, arginine is most conserved, as is glutamate in motif III. These two residues have been shown to be important in binding glycerol-3-phosphate. Motif IV is highly hydrophobic and has been suggested to be involved in acyl-CoA binding. The proline in motif IV appears to be most conserved (FIG. 1A). The substitution of serine for proline in motif IV can lead to a 2-5 fold higher Km for palmitoyl-CoA.

A Kyte-Doolittle hydropathy analysis of the amino acid sequence of the TpGPAT revealed several hydrophobic domains (data not shown). Protein topology analysis with the algorithms (TMHHM, SOSUI, and TMAP) predicted 5 transmembrane domains, with 2 of them close to the N-terminus and 3 close to the C-terminus (FIG. 1C). This topology strongly suggests that TpGPAT has the membrane-bound nature like other ER- or mitochondria-based GPATs from lower and higher eukaryotes. As shown in FIG. 1C, the N- and C-termini of TpGPAT are located on the cytosolic (outside) and lumenal (inside) sides, respectively. In the middle, a long stretch of more than 400 amino acids encompassing all 4 acyltransferase motifs is exposed to the cytosol, which allows the binding and catalysis of the substrates to take place in the same space (FIG. 1C).

### Example IV

### Heterologous Expression of TpGPAT in the Yeast gat1 Mutant

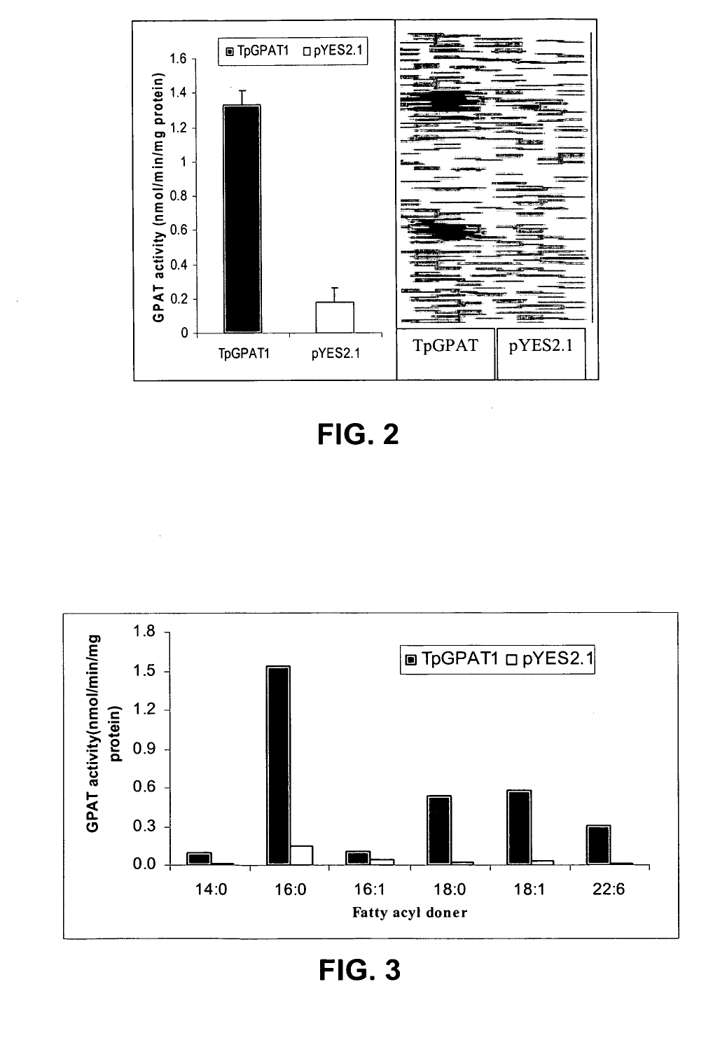

To determine the functionality of the protein encoded by the TpGPAT, the full-length coding region of TpGPAT was cloned into a yeast expression vector pYES2.1/V5-His-TOPO under the control of the galactose-inducible GAL1 promoter, and the construct was used to transform a GPAT-deficient yeast strain, gat1 (EUROSCARF accession no. Y15983). The gat1 cells harboring an empty pYES2.1 vector were used as a control. The microsomal membrane fractions prepared from lysates of the induced yeast cells were assayed for GPAT activity using ¹⁴C-labelled glycerol-3-phosphate as acceptor, and palmitoyl (16:0)-CoA as acyl donor. Under our assay conditions, expression of the TpGPAT in yeast gad mutant resulted in a restoration of TpGPAT function with about seven-fold higher activity than that found in control cells transformed with empty pYES2.1 vector (FIG. 2). When different unlabeled acyl-CoAs were used as acyl donors, it was shown that the recombinant TpGPAT protein possesses the highest activity toward 16:0. In contrast, the GPAT activities toward other fatty acyl donors including 14:0-, 16:0-, 16:1-, 18:0-, 18:1-, and 22:6 DHA)-CoA are much lower. (FIG. 3).

### Example V

### Fatty Acids Profile in Yeast gat1 Mutant Expressing TpGPAT

To investigate if the over-expression of TpGPAT has any effect on the fatty acids composition of yeast lipids in vivo, the gall cells harboring TpGPAT or an empty vector as control were induced for 3 days by galactose at 28° C. The total lipids was extracted and then separated into triacylglycerols (TAGS) and phospholipids by TLC for the analysis of fatty acid composition. As shown in Table 1, the expression of TpGPAT in gad resulted in a significant change in the fatty acid composition of both TAG and phospholipids as compared to the control with empty vector. Approximately 12% and 18% increase of 16:0 in phospholipids and triacylglycerols, respectively, turned 16:0 into the most dominant fatty acid in the TpGPAT expressing yeast cells, instead of 16:1 fatty acid in the mutant cells harboring the empty vector. Conversely, the unsaturated fatty acids, 16:1 and 18:1, were reduced to 15% and 21% in these two lipid species. In addition, a slight increase of 3-4% was observed for 18:0 in both the lipid species. Collectively, the results strongly support the finding in the in vitro substrate specificity assay that 16:0 is the most preferred substrate for TpGPAT.

### Example VI

### Incorporation of EPA/DHA into Lipids in Yeast gat1 Mutant Expressing TpGPAT

Since glycerolipids of T. pseudonana contain a high percentage of a very-long chain polyunsaturated fatty acid (VLGPUFA), EPA (20:5n3), we tested if the expression of the TpGPAT gene could increase the accumulation of EPA and DHA in yeast glycerolipids.

Yeast gat1 strain transformed with TpGPAT or empty vector pYES2.1 was grown in the presence of EPA or DHA, while being induced by galactose. Triacylglycerols (TAGS) and phospholipids from the 3-day culture were extracted and analyzed by gas chromatography for fatty acid composition. The expression of TpGPAT in gat1 did not have much impact on the incorporation of LCPUFAs into either TAG or phospholipids as compared to the empty vector control (Table 2). It was not clear if this were due to the low GPAT activity for these VLCPUFAs or the lack of EPA and DHA-CoA in the cells.

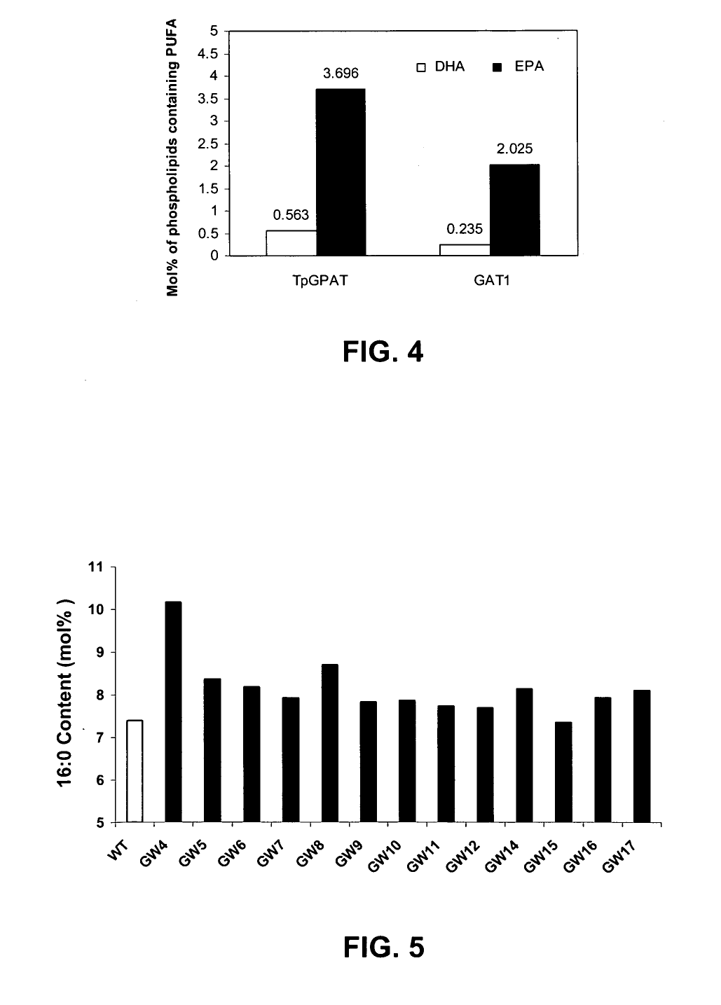

To further determine the role of TpGPAT in the incorporation of VLGPUFAs into glycerolipids, yeast gat1 strain transformed with TpGPAT or GAT1 under the control of the GAL1 promoter was grown in the presence of EPA (20:5) or DHA (22:6) upon induction of the genes. Phospholipids were extracted and subjected to lipidomic analysis using a tandem mass spectrometer (testing conducted by the Kansas Lipidomics Research Center). The results revealed that PC38:7, PC38:6, PC38:5, PC36:6, PC36:5, LPC20:5, LPC22:6, PE38:6, PE36:5, LPE20:5, LPE22:6, and PI36:5 from TpGPAT-expressing cells contain much higher EPA or DHA than those from GAT1-expressing cells. As shown in FIG. 4, the molar percentage of PUFA in phospholipids from TpGPAT-expressing yeast cells contain 0.563% DHA 0.10 and 3.696% EPA respectively, while the GAT1-expressing yeast cells contain only 0.235% DHA and 2.025% EPA respectively. This experiment directly compares the activity of expressed TpGPAT with GAT1, and the result suggests that TpGPAT enhances PUFA incorporation into the sn-1 position of a glycerol backbone and/or that LPA synthesized through a TpGPAT-mediated initial reaction is a more favorable acceptor for PUFA. It was thus concluded that TpGPAT has a role in controlling PUFA accumulation in glycerolipid.

DISCUSSION: GPAT catalyzes the first (and potentially rate-limiting) step in glycerolipid biosynthesis in eukaryotes. However, clear evidence that GPAT plays a role in determining the fatty acid composition of glycerolipids was lacking. Owing to the membrane-bound nature, no GPAT has been purified to an apparent purity sufficient for accurate in vitro biochemical assay. Studies using partially purified membrane-bound GPATs, which are often contaminated with other fatty acyltransferases, suggested a broad range of fatty acids as acyl donors for this enzyme. Nonetheless, it would be a reasonable assumption that substrate specificity of GPATs varies among different species. This assumption is supported by the present study revealing that TpGPAT shows high specificity for palmitate as fatty acyl donor in both in vitro and in vivo assays.

In order to assess the substrate specificity TpGPAT, we conducted heterologous expression in yeast gall mutant, which possesses very low GPAT activity. This experiment restored GPAT activity, demonstrating close to seven-fold increase in enzyme activity when compared to gat1 cells harboring empty pYES2.1 vector (FIG. 2). Hence, the substrate specificity resulting from this heterologous system can be ascribed to TpGPAT because the interference caused by the native Gat2p in yeast is relatively low. Our in vitro assays revealed that TpGPAT is highly specific for saturated C16 fatty acids although low activities toward other fatty acids were also detected.

This finding is further consolidated by examining the fatty acid composition in the gat1 cells expressing TpGPAT. Heterologous expression of TpGPAT led to increases of palmitate in phospholipids and triacylglycerols by approximately 12% and 18%, respectively, in the gat1 cells (Table 1). Accordingly, unsaturated fatty acids, mainly 16:1 and 18:1, dropped by 15% and 21% in phospholipids and triacylglycerols (Table 1). This unique substrate specificity of TpGPAT may constitute one of main factors controlling the fatty acid profile of T. pseudonana in which fatty acid 16:0 is predominant, accounting for up to 28% and 36% of fatty acids in total lipids and triacylglycerols, respectively.

It is noteworthy that the marine diatom T. pseudonana has a very low level of C18 fatty acids. (Tonon et al., 2002; Tonon et al., 2005). One possible explanation is that this species has much lower steady levels of 18:0-CoA and 18:1-CoA pools relative to 16:0-CoA, 16:1-CoA and 205-CoA, as suggested by Tonon et al. (2005). However, an important aspect of fatty acyl pool is its dynamic nature. If 18:0-CoA and 18:1-CoA pools are not channeled away by fatty acyltransferases, they can be converted to other fatty acid pools through processes such as fatty acid elongation and β-oxidation. Thus, we suggest that fatty acyltransferases not only directly control the fatty acid composition in glycerolipids through their preferential incorporation of fatty acids into glycerol backbone, but also indirectly monitor fatty acyl pools that they use.

### Example VII

Amino acid sequence of prophetic GPAT I
(SEQ ID NO: 3)
MGVEKKGTMMSELDYTKAQLAFFYIVVLLSLDMLNPVKIFLHVFPAIKS
WHIATFAIACMSYIFIVNLRELLYFATKVFFHSILSIFFNDVTVVGREN
IPSHGPVIFTSNHANQFMDGLMIMCTCQRTISYLVADKSWNRPIIGHLA
WMMGGVPVKRAQDSACKGTGKISIDLNALAGSDAVINVVGKGTSFTSQI
KAGDKIRLPNNAIGIKVESIESDESMSLKVEDGVAEVLSSHPFPEYVTY
DILPRIDQKDVYQNVLEKLASGGTIGIFPEGGSHDRTDLLPLKVGVALI
AYSELEKDGINVPIVPVGLNYFRAHRFRGKATVEFGSPTYIEPSTLADY
KKGGADKRRVCNDLLARIENSMRSVIVSVPDFETLQTIHAARRLYRQDG
RNETAEQRQDMGRRFAEGYKRVLLQLGGEPPEEWLSLQSRILAYQKELN
ELGIRDYQVVGLDHEEVELGSESQGHSKADTVLHRMNVFGHIVHLFVIA
VLAALPAMLLNLPVGLASRIYSNRRRKVALAASKVKVKGYDVMLSERVL
ACIVLVPSLWVVYGLLLSLFTSLDGPSLAVCFTCFPLFSYWSIMATESG
MVDIKDLRPYVMRMIPSARRYDVSSDATRKALRSDLRAMIKKIGPSLG
DIYYEKDLNWQKIQMETKRMSMEELDPAQKDEAAKKEE

### Example VIII

Amino acid sequence of prophetic GPAT II
(SEQ ID NO: 4)
MGVEKKGTMMSELDYTKAQLAFFYIVVLLSLDMLNPVKIFLHVFPAIKS
WHIATFAIACMSYIFIVNLRELLYFATKVFFHSILSIFFNDVTVVGREN
IPSHGPVIFTSNHANQFMDGLMIMCTCQRTISYLVADKSWNRPIIGHLA
WMMGGVPVKRAQDSACKGTGKISIDLNALAGSDAVINVVGKGTSFTSQI
KAGDKIRLPNNAIGIKVESIESDESMSLKVEDGVAEVLSSHPFPEYVTY
DILPRIDQKDVYQNVLEKLASGGTIGIFPEGGSHDRTDLLPLKVGVALI
AYSELEKDGINVPIVPVGLNYFRAHRFRGKATVEFGSPTYIEPSTLADY
KKGGADKRRVCNDLLARIENSMRSVIVSVPDFETLQTIHAARRLYRQDG
RNETAEQRQDMGRRFAEGYKRVLLQLGGEPPEEWLSLQSRILAYQKELN
ELGIRDYQVVGLDHEEVELGSESQGHSKADTVLHRMNVFGHIVHLFVIA
VLAALPAMLLNLPVGLASRIYSNRRRKVALAASKVKVKGYDVMLSERVL
ACIVLVPSLWVVYGLLLSLFTSLDGPSLAVCFTCFPLFSYWSIMATESG
MVDIKDLRPYVMRMIPSARRRKTSELNRRKALRSDLRAMIKKIGPSLGD
IYYEKDLNWQKIQMETKRMSMEELDPAQKDEAAKKEE

### Example IX

Amino acid sequence of prophetic GPAT III
(SEQ ID NO: 5)
MGVEKKGTMMMELWPGAWTALLQLLLLLLSTLWFCSSSAKYFFKMAFYS
WHIATFAIACMSYIFIVNLRELLYFATKVFFHSILSIFFNDVTVVGREN
IPSHGPVIFTSNHANQFMDGLMIMCTCQRPVRFLMAEKSFQRPVIGHLA
WMMGGVPVKRAQDSACKGTGKISIDLNALAGSDAVINVVGKGTSFTSQI
KAGDKIRLPNNAISWGVPGNAKCRAQVSRITSDEEVELSSHPFPEYVTY
DILPRIDQKDVYQNVLEKLASGGTIGIFPEGGSHDRTDLLPLKVGVALI
AYSELEKDGINVPIVPVGLNYFRAHRFRGKATVEFGSPTYIEPSTLADY
KKGGADKRRVCNDLLARIENSMRSVIVSVPDFETLQTIHAARRLYRQDG
RNETAEQRQDMGRRFAEGYKRVLLQLGGEPPEEWLSLQSRILAYQKELN
ELGIRDYQVVGLDHEEVELGSESQGHSKADTVLHRMNVFGHIVHLFVIA
VLAALPAMLLNLPVGLASRIYSNRRRKVALAASKVKVKGYDVMLSERVL
ACIVLVPSLWVVYGLLLSLFTSLDGPSLAVCFTCFPLFSYWSIMATESG
MVDIKDLRPYVMRMIPSARRRLAALPATRKALRSDLRARSRSSGPSLGD
IYYEKDLNWQKIQMETKRMSMEELDPAQKDEAAKKEE

### Example X

Amino acid sequence of prophetic GPAT IV
(SEQ ID NO: 6)
MGVEKKGTMMSELDYTKAQLAFMPAPKLTEKFASSKKSTQKHVFPAIKS
WHIATFAIACMSYIFIVNLRELLYFATKVFFHSILSIFFNDVTVVGREN
IPSHGPVIFTSNHANQFMDGLMIMCTCQRPVRFLMAEKSFQRPVIGHLA
WMMGGVPVKRAQDSACKGTGKISIDLNALAGSDAVINVVGKGTSFTSQI
KAGDKIRLPNNAISWGVPGNAKCRAQVSRITSDEEVELSSHPFPEYVTY
DILPRIDQKDVYQNVLEKLASGGTIGIFPEGGSHDRTDLLPLKVGVALI
AYSELEKDGINVPIVPVGLNYFRAHRFRGKATVEFGSPTYIEPSTLADY
KKGGADKRRVCNDLLARIENSMRSVIVSVPDFETLQTIHAARRLYRQDG
RNETAEQRQDMGRRFAEGYKRVLLQLGGEPPEEWLSLQSRILAYQKELN
ELGIRDYQVVGLDHEEVELGSESQGHSKADTVLHRMNVFGHIVHLFVIA
VLAALPAMLLNLPVGLASRIYSNRRRKVALAASKVKVKGYDVMLSERVL
ACIVLVPSLWVVYGLLLSLFTSLDGPSLAVCFTCFPLFSYWSIMATESG
MVDIKDLRPYVMRMIPSARRRLAALPATRKALRSDLRARSRSSGPSLGD
IYYEKDLNWQKIQMETKRMSMEELDPAQKDEAAKKEE

### Example XI

Amino acid sequence of prophetic GPAT V
(SEQ ID NO: 7)
MGVEKKGTMMSELDYTKAQLAFFYIVVLLSLDMLNPVKIFLHVFPAIKS
WHIATFAIACMSYIFIVNLRELLYFATKVFFHSILSIFFNDVTVVGREN
IPSHGPVIFTSNHANQFMDGLMIMCTCQRTISYLVADKSWNRPIIGHLA
WMMGGVPVKRAQDSACKGTGKISIDLNALAGSDAVINVVGKGTSFTSQI
KAGDKIRLPNNAIGIKVESIESDESMSLKVEDGVAEVLSSHPFPEYVTY
DILPRIDQKDVYQNVLEKLASGGTIGIFPEGGSHDRTDLLPLKVGVALI
AYSELEKDGINVPIVPVGLNYFRAHRFRGKATVEFGSPTYIEPSTLADY
KKGGADKRRVCNDLLARIENSMRSVIVSVPDFETLQTIHAARRLYRQDG
RNETAEQRQDMGRRFAEGYKRVLLQLGGEPPEEWLSLQSRILAYQKELN
ELGIRDYQVVGLDHEEVELGSESQGHSKADTVLHRMNVFGHIVHLFVIA
VLAALPAMLLNLPVGLASRIYSNRRRKVALAASKVKVKGYDVMLSERVL
ACIVLVPSLWVVYGLLLSLFTSLDGPSLAVCFTCFPLFSYWSIMATEIG
MDGFKSLRPLVLSLTSPARRRLAALPATRKALRSDLRAMIKKIGPSLGD
IYYEKDLNWQKIQMETKRMSMEELDPAQKDEAAKKEE

### Example XII

A first prophetic nucleotide
sequence of T. pseudonana GPAT
(SEQ ID NO: 8)
ATGGGUGTCGAGAAAAAAGGAACGATGATGTCCGAGTTGGACTATACGA
AGGCACAACTCGCCTTCTTCTACATCGTCGTCCTTCTATCACTCGATAT
GCTCAACCCAGTCAAGATCTTTTTACACGTCTTTCCTGCAATTAAGTCA
TGGCACATCGCGACATTTGCAATTGCCTGCATGTCATACATCTTCATCG
TGAACTTGAGGGAACTGCTATACTTCGCTACCAAGGTCTTCTTCCATTC
AATCCTATCAATCTTTTTCAACGACGTGACCGTGGTTGGCAGAGAGAAT
ATCCCGAGCCATGGCCCTGTTATCTTTACCTCCAACCACGCTAATCAGT
TTATGGATGGGTTGATGATTATGTGTACTTGCCAAAGGACGATCTCGTA
TCTTGTAGCAGACAAGTCTTGGAATAGACCAATCATTGGGCATCTGGCT
TGGATGATGGGGGGAGTGCCAGTCAAACGTGCACAAGATAGTGCCTGTA
AAGGAACTGGAAAAATCAGCATTGACTTGAACGCTCTCGCGGGATCGGA
TGCAGTCATCAATGTCGTTGGAAAGGGAACATCGTTCACGTCTCAGATA
AAAGCCGGGGATAAGATTCGCCTACCAAACAATGCAATCGGCATCAAAG
TTGAATCTATCGAAAGTGATGAATCAATGTCGCTCAAAGTGGAAGATGG
TGTGGCTGAAGTATTATCATCCCATCCATTTCCTGAGTACGTCACATAC
GATATTCTGCCTCGAATTGATCAGAAGGACGTCTACCAAAATGTACTGG
AGAAACTAGCATCAGGCGGGACGATTGGAATCTTTCCAGAGGGTGGCTC
CCACGATAGGACTGACTTGCTCCCATTGAAAGTTGGTGTGGCACTCATT
GCATACTCGGAACTTGAAAAGGATGGAATCAACGTGCCGATTGTCCCAG
TTGGATTGAACTACTTTCGGGCTCATCGCTTCCGTGGCAAAGCAACTGT
TGAGTTTGGTTCTCCAACTTATATTGAACCATCGACACTTGCAGACTAC
AAAAAGGGAGGTGCCGATAAGCGACGCGTTTGCAATGATCTTTTGGCTC
GTATTGAGAACAGTATGAGATCTGTCATTGTGTCGGTGCCCGACTTTGA
AACACTTCAGACCATCCATGCTGCGAGGAGGCTATACAGACAAGATGGC
AGAAACGAAACTGCTGAACAAAGGCAGGACATGGGCAGGCGATTTGCAG
AGGGGTACAAACGCGTTCTTCTCCAATTAGGAGGAGAGCCGCCAGAGGA
GTGGCTCAGTCTGCAGTCGAGGATATTGGCGTACCAAAAGGAATTGAAC
GAGCTCGGTATCAGGGACTATCAGGTTGTTGGTCTTGATCACGAGGAGG
TGGAACTCGGTTCAGAGTCGCAAGGTCATTCCAAAGCAGATACTGTTCT
TCACAGGATGAATGTGTTTGGACACATCGTTCATCTGTTTGTCATTGCG
GTCTTGGCAGCACTGCCAGCTATGTTGTTAAACCTTCCAGTTGGGTTGG
CATCACGAATCTACTCCAATCGTAGGCGAAAGGTTGCATTGGCAGCATC
AAAAGTGAAGGTGAAGGGATATGATGTGATGCTCTCAGAACGTGTACTG
GCATGTATCGTTCTCGTTCCTTCTCTGTGGGTAGTGTATGGATTGCTTC
TCTCCCTGTTCACCTCTCTCGACGGACCGTCACTTGCTGTATGCTTCAC
CTGCTTTCCTTTGTTCTCATATTGGAGTATTATGGCTACGGAATCAGGA
ATGGTTGATATCAAAGATTTGAGGCCGTACGTTATGAGAATGATTCCAT
CAGCACGACGCAGGTTAGCAGCATTGCCAGCAACGAGGAAGGCTCTCCG
GTCGGATCTTAGAGCAATGATTAAGAAGATTGGCCCCAGCTTGGGTGAT
ATTTACTACGAAAAGGACTTGAACTGGCAGAAGATTCAAATGGAGACGA
AGAGGATGTCAATGGAGGAGTTGGATCCAGCTCAAAAAGACGAAGCAGC
GAAGAAGGAAGAGTAA

### Example XIII

A second prophetic nucleotide
sequence of T. pseudonana GPAT
(SEQ ID NO: 9)
ATGGGUGTCGAGAAAAAAGGAACGATGATGTCCGAGTTGGACTATACGA
AGGCACAACTCGCCTTCTTCTACATCGTCGTCCTTCTATCACTCGATAT
GCTCAACCCAGTCAAGATCTTTTTACACGTCTTTCCTGCAATTAAGTCA
TGGCACATCGCGACATTTGCAATTGCCTGCATGTCATACATCTTCATCG
TGAACTTGAGGGAACTGCTATACTTCGCTACCAAGGTCTTCTTCCATTC
AATCCTATCAATCTTTTTCAACGACGTGACCGTGGTTGGCAGAGAGAAT
ATCCCGAGCCATGGCCCTGTTATCTTTACCTCCAACCACGCTAATCAGT
TTATGGATGGGTTGATGATTATGTGTACTTGCCAAAGGACGATCTCGTA
TCTTGTAGCAGACAAGTCTTGGAATAGACCAATCATTGGGCATCTGGCT
TGGATGATGGGGGGAGTGCCAGTCAAACGTGCACAAGATAGTGCCTGTA
AAGGAACTGGAAAAATCAGCATTGACTTGAACGCTCTCGCGGGATCGGA
TGCAGTCATCAATGTCGTTGGAAAGGGAACATCGTTCACGTCTCAGATA
AAAGCCGGGGATAAGATTCGCCTACCAAACAATGCAATCGGCATCAAAG
TTGAATCTATCGAAAGTGATGAATCAATGTCGCTCAAAGTGGAAGATGG
TGTGGCTGAAGTATTATCATCCCATCCATTTCCTGAGTACGTCACATAC
GATATTCTGCCTCGAATTGATCAGAAGGACGTCTACCAAAATGTACTGG
AGAAACTAGCATCAGGCGGGACGATTGGAATCTTTCCAGAGGGTGGCTC
CCACGATAGGACTGACTTGCTCCCATTGAAAGTTGGTGTGGCACTCATT
GCATACTCGGAACTTGAAAAGGATGGAATCAACGTGCCGATTGTCCCAG
TTGGATTGAACTACTTTCGGGCTCATCGCTTCCGTGGCAAAGCAACTGT
TGAGTTTGGTTCTCCAACTTATATTGAACCATCGACACTTGCAGACTAC
AAAAAGGGAGGTGCCGATAAGCGACGCGTTTGCAATGATCTTTTGGCTC
GTATTGAGAACAGTATGAGATCTGTCATTGTGTCGGTGCCCGACTTTGA
AACACTTCAGACCATCCATGCTGCGAGGAGGCTATACAGACAAGATGGC
AGAAACGAAACTGCTGAACAAAGGCAGGACATGGGCAGGCGATTTGCAG
AGGGGTACAAACGCGTTCTTCTCCAATTAGGAGGAGAGCCGCCAGAGGA
GTGGCTCAGTCTGCAGTCGAGGATATTGGCGTACCAAAAGGAATTGAAC
GAGCTCGGTATCAGGGACTATCAGGTTGTTGGTCTTGATCACGAGGAGG
TGGAACTCGGTTCAGAGTCGCAAGGTCATTCCAAAGCAGATACTGTTCT
TCACAGGATGAATGTGTTTGGACACATCGTTCATCTGTTTGTCATTGCG
GTCTTGGCAGCACTGCCAGCTATGTTGTTAAACCTTCCAGTTGGGTTGG
CATCACGAATCTACTCCAATCGTAGGCGAAAGGTTGCATTGGCAGCATC
AAAAGTGAAGGTGAAGGGATATGATGTGATGCTCTCAGAACGTGTACTG
GCATGTATCGTTCTCGTTCCTTCTCTGTGGGTAGTGTATGGATTGCTTC
TCTCCCTGTTCACCTCTCTCGACGGACCGTCACTTGCTGTATGCTTCAC
CTGCTTTCCTTTGTTCTCATATTGGAGTATTATGGCTACGGAATCAGGA
ATGGTTGATATCAAAGATTTGAGGCCGTACGTTATGAGAATGATTCCAT
CAGCACGACGCAGGTTAGCAGCATTGCCAGCAACGAGGAAGGCTCTCCG
GTCGGATCTTAGAGCAATGATTAAGAAGATTGGCCCCAGCTTGGGTGAT
ATTTACTACGAAAAGGACTTGAACTGGCAGAAGATTCAAATGGAGACGA
AGAGGATGTCAATGGAGGAGTTGGATCCAGCTCAAAAAGACGAAGCAGC
GAAGAAAGAGGAATAA

### Example XIV

A nucleotide sequence of prophetic GPATI
(SEQ ID NO: 10)
ATGGGCGTTGAAAAGAAGGGCACAATGATGTCCGAGTTGGACTATACGA
AGGCACAACTCGCCTTCTTCTACATCGTCGTCCTTCTATCACTCGATAT
GCTCAACCCAGTCAAGATCTTTTTACACGTCTTTCCTGCAATTAAGTCA
TGGCACATCGCGACATTTGCAATTGCCTGCATGTCATACATCTTCATCG
TGAACTTGAGGGAACTGCTATACTTCGCTACCAAGGTCTTCTTCCATTC
AATCCTATCAATCTTTTTCAACGACGTGACCGTGGTTGGCAGAGAGAAT
ATCCCGAGCCATGGCCCTGTTATCTTTACCTCCAACCACGCTAATCAGT
TTATGGATGGGTTGATGATTATGTGTACTTGCCAAAGGACGATCTCGTA
TCTTGTAGCAGACAAGTCTTGGAATAGACCAATCATTGGGCATCTGGCT
TGGATGATGGGGGGAGTGCCAGTCAAACGTGCACAAGATAGTGCCTGTA
AAGGAACTGGAAAAATCAGCATTGACTTGAACGCTCTCGCGGGATCGGA
TGCAGTCATCAATGTCGTTGGAAAGGGAACATCGTTCACGTCTCAGATA
AAAGCCGGGGATAAGATTCGCCTACCAAACAATGCAATCGGCATCAAAG
TTGAATCTATCGAAAGTGATGAATCAATGTCGCTCAAAGTGGAAGATGG
TGTGGCTGAAGTATTATCATCCCATCCATTTCCTGAGTACGTCACATAC
GATATTCTGCCTCGAATTGATCAGAAGGACGTCTACCAAAATGTACTGG
AGAAACTAGCATCAGGCGGGACGATTGGAATCTTTCCAGAGGGTGGCTC
CCACGATAGGACTGACTTGCTCCCATTGAAAGTTGGTGTGGCACTCATT
GCATACTCGGAACTTGAAAAGGATGGAATCAACGTGCCGATTGTCCCAG
TTGGATTGAACTACTTTCGGGCTCATCGCTTCCGTGGCAAAGCAACTGT
TGAGTTTGGTTCTCCAACTTATATTGAACCATCGACACTTGCAGACTAC
AAAAAGGGAGGTGCCGATAAGCGACGCGTTTGCAATGATCTTTTGGCTC
GTATTGAGAACAGTATGAGATCTGTCATTGTGTCGGTGCCCGACTTTGA
AACACTTCAGACCATCCATGCTGCGAGGAGGCTATACAGACAAGATGGC
AGAAACGAAACTGCTGAACAAAGGCAGGACATGGGCAGGCGATTTGCAG
AGGGGTACAAACGCGTTCTTCTCCAATTAGGAGGAGAGCCGCCAGAGGA
GTGGCTCAGTCTGCAGTCGAGGATATTGGCGTACCAAAAGGAATTGAAC
GAGCTCGGTATCAGGGACTATCAGGTTGTTGGTCTTGATCACGAGGAGG
TGGAACTCGGTTCAGAGTCGCAAGGTCATTCCAAAGCAGATACTGTTCT
TCACAGGATGAATGTGTTTGGACACATCGTTCATCTGTTTGTCATTGCG
GTCTTGGCAGCACTGCCAGCTATGTTGTTAAACCTTCCAGTTGGGTTGG
CATCACGAATCTACTCCAATCGTAGGCGAAAGGTTGCATTGGCAGCATC
AAAAGTGAAGGTGAAGGGATATGATGTGATGCTCTCAGAACGTGTACTG
GCATGTATCGTTCTCGTTCCTTCTCTGTGGGTAGTGTATGGATTGCGCT
TCTCTCCCTGTTCACCTCTCTCGACGGACCGTCACTTGCTGTATGCTTC
ACCTGCTTTCCTTTGTTCTCATATTGGAGTATTATGGCTACGGAATCAG
GAATGGTTGATATCAAAGATTTGAGGCCGTACGTTATGAGAATGATTCC
ATCAGCACGACGCTACGATGTATCATCGGATGCAACGAGGAAGGCTCTC
CGGTCGGATCTTAGAGCAATGATTAAGAAGATTGGCCCCAGCTTGGGTG
ATATTTACTACGAAAAGGACTTGAACTGGCAGAAGATTCAAATGGAGAC
GAAGAGGATGTCAATGGAGGAGTTGGATCCAGCTCAAAAAGACGAAGCA
GCGAAGAAGGAAGAGTAA

### Example XV

A nucleotide sequence of prophetic GPATII
(SEQ ID NO: 11)
ATGGGCGTTGAAAAGAAGGGCACAATGATGTCCGAGTTGGACTATACGA
AGGCACAACTCGCCTTCTTCTACATCGTCGTCCTTCTATCACTCGATAT
GCTCAACCCAGTCAAGATCTTTTTACACGTCTTTCCTGCAATTAAGTCA
TGGCACATCGCGACATTTGCAATTGCCTGCATGTCATACATCTTCATCG
TGAACTTGAGGGAACTGCTATACTTCGCTACCAAGGTCTTCTTCCATTC
AATCCTATCAATCTTTTTCAACGACGTGACCGTGGTTGGCAGAGAGAAT
ATCCCGAGCCATGGCCCTGTTATCTTTACCTCCAACCACGCTAATCAGT
TTATGGATGGGTTGATGATTATGTGTACTTGCCAAAGGACGATCTCGTA
TCTTGTAGCAGACAAGTCTTGGAATAGACCAATCATTGGGCATCTGGCT
TGGATGATGGGGGGAGTGCCAGTCAAACGTGCACAAGATAGTGCCTGTA
AAGGAACTGGAAAAATCAGCATTGACTTGAACGCTCTCGCGGGATCGGA
TGCAGTCATCAATGTCGTTGGAAAGGGAACATCGTTCACGTCTCAGATA
AAAGCCGGGGATAAGATTCGCCTACCAAACAATGCAATCGGCATCAAAG
TTGAATCTATCGAAAGTGATGAATCAATGTCGCTCAAAGTGGAAGATGG
TGTGGCTGAAGTATTATCATCCCATCCATTTCCTGAGTACGTCACATAC
GATATTCTGCCTCGAATTGATCAGAAGGACGTCTACCAAAATGTACTGG
AGAAACTAGCATCAGGCGGGACGATTGGAATCTTTCCAGAGGGTGGCTC
CCACGATAGGACTGACTTGCTCCCATTGAAAGTTGGTGTGGCACTCATT
GCATACTCGGAACTTGAAAAGGATGGAATCAACGTGCCGATTGTCCCAG
TTGGATTGAACTACTTTCGGGCTCATCGCTTCCGTGGCAAAGCAACTGT
TGAGTTTGGTTCTCCAACTTATATTGAACCATCGACACTTGCAGACTAC
AAAAAGGGAGGTGCCGATAAGCGACGCGTTTGCAATGATCTTTTGGCTC
GTATTGAGAACAGTATGAGATCTGTCATTGTGTCGGTGCCCGACTTTGA
AACACTTCAGACCATCCATGCTGCGAGGAGGCTATACAGACAAGATGGC
AGAAACGAAACTGCTGAACAAAGGCAGGACATGGGCAGGCGATTTGCAG
AGGGGTACAAACGCGTTCTTCTCCAATTAGGAGGAGAGCCGCCAGAGGA
GTGGCTCAGTCTGCAGTCGAGGATATTGGCGTACCAAAAGGAATTGAAC
GAGCTCGGTATCAGGGACTATCAGGTTGTTGGTCTTGATCACGAGGAGG
TGGAACTCGGTTCAGAGTCGCAAGGTCATTCCAAAGCAGATACTGTTCT
TCACAGGATGAATGTGTTTGGACACATCGTTCATCTGTTTGTCATTGCG
GTCTTGGCAGCACTGCCAGCTATGTTGTTAAACCTTCCAGTTGGGTTGG
CATCACGAATCTACTCCAATCGTAGGCGAAAGGTTGCATTGGCAGCATC
AAAAGTGAAGGTGAAGGGATATGATGTGATGCTCTCAGAACGTGTACTG
GCATGTATCGTTCTCGTTCCTTCTCTGTGGGTAGTGTATGGATTGCTTC
TCTCCCTGTTCACCTCTCTCGACGGACCGTCACTTGCTGTATGCTTCAC
CTGCTTTCCTTTGTTCTCATATTGGAGTATTATGGCTACGGAATCAGGA
ATGGTTGATATCAAAGATTTGAGGCCGTACGTTATGAGAATGATTCCAT
CAGCACGACGCCGTAAAACATCAGAGTTAAACAGGAGGAAGGCTCTCCG
GTCGGATCTTAGAGCAATGATTAAGAAGATTGGCCCCAGCTTGGGTGAT
ATTTACTACGAAAAGGACTTGAACTGGCAGAAGATTCAAATGGAGACGA
AGAGGATGTCAATGGAGGAGTTGGATCCAGCTCAAAAAGACGAAGCAGC
GAAGAAGGAAGAGTAA

### Example XVI

### Preparation of Nucleic Acid Molecules for Use in Plant Transformation

The full length coding region of the TpGPAT gene was amplified using pfu DNA polymerase and primers designed with two restriction sites (KpnI and XbaI) added for subsequent cloning. Then, the PCR product was digested and inserted in a plant transformation vector (pSE129A) under the control of a seed-specific promoter (Napin). The binary vector was introduced by electroporation into Agrobacterium tumefaciens strain GV3101 containing helper plasmid pMP90 (Koncz and Schell, 1986).

### Example XVII

### Use of TpGPAT to Increase Yield and Modify the Composition of Oilseed Produced from Two Oilseed Crops; Arabidopsis thaliana and Brassica napus

Wild-type A. thaliana (ecotype Columbia) were subjected to Agrobacterium-mediated transformation by the floral dip method using the A. tumefaciens carrying the TpGPAT gene under the control of the Napin promoter produced in Example XVI. (Clough and Bent, 1998). Seeds from Agrobacterium transformed plants were then plated on selective medium and kanamycin resistant T1 plants were transferred to soil and their genotype characterized. DNA was isolated from 150 mg of Arabidopsis leaf material. Plants that contained the insertion (napin:TpGPAT:nos) cassette were identified by PCR amplification of genomic DNA, and the T2 seeds were harvested for fatty acid composition analysis. More than half of the identified transgenic lines (GW) showed an increase of 16:0 compared to the non-transformed control (WT). (FIG. 5). Since not all T2 seeds are homozygous for the transgene, it is anticipated that homozygous T3 seeds will have exhibit a further change in 16:0 compared to wild-type seeds. Total oil content in T2 seeds from TpGPAT transgenic Arabidopsis lines also increased. (FIG. 7). The absolute increase values ranged from 0.5% to 8.6%.

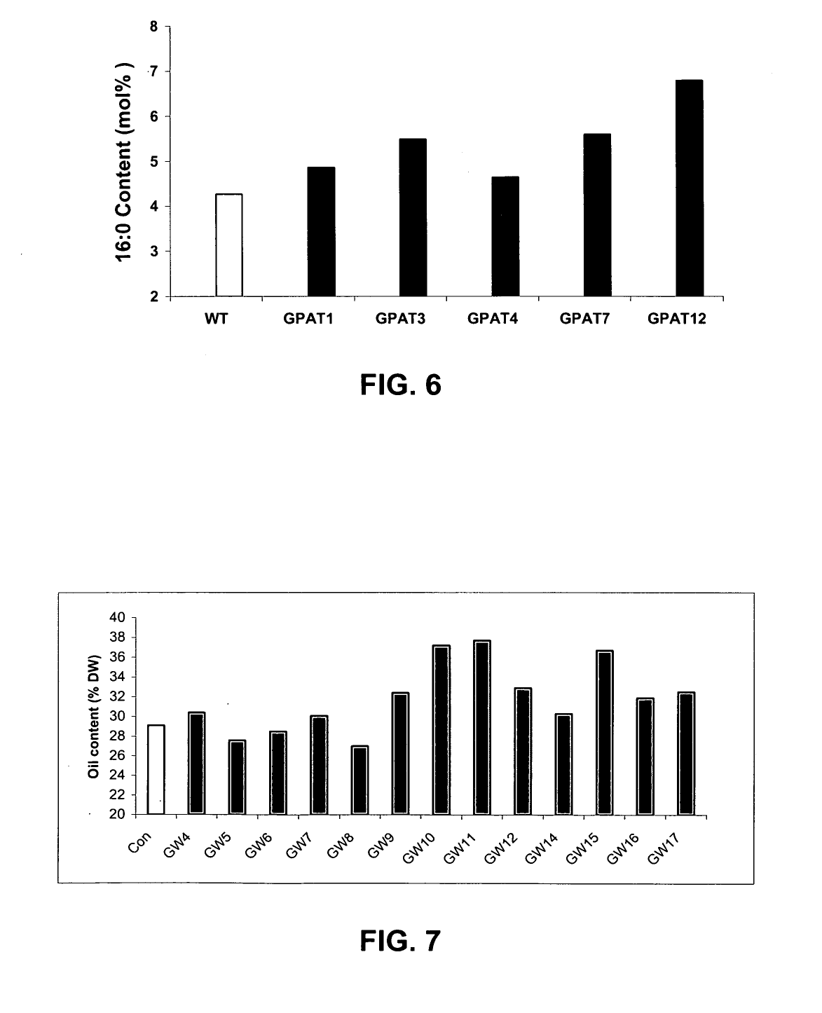

Brassica napus 5-day-old hypocotyls were also transformed with Agrobacterium containing the TpGPAT/pSE129A construct. Transgenic T0 plants were regenerated, selected for resistance to kanamycin and grown in soil. Individual plants were bagged to allow self-pollination. Presence of the TpGPAT and Kan genes in the resistant plants was verified by PCR with the appropriate primers in 18 independent events. T1 seeds from the first set of 5 transgenic events were harvested and analyzed. (FIG. 6). T1 seeds showed increased 16:0 percentage with 4.64-6.67% among the transgenic plants versus 4.27-4.64% for wild-type plants. Given that T1 B. napus seeds are a segregation population, it is expected that the effect of the TpGPAT gene on controlling 16:0 level in T2 homozygous seeds will be increased.

### Example XVIII

### Use of TpGPAT1 to Produce Ethanol and Biodiesel

U.S. Pat. No. 5,578,472 to Ueda et al. and U.S. Pat. No. 7,135,308 to Bush et al., the contents of the entirety of each of which are incorporated herein by this reference, describe a process for the production of ethanol by harvesting starch-accumulating filament-forming or colony-forming algae to form a biomass, initiating cellular decay of the biomass in a dark and anaerobic environment, fermenting the biomass in the presence of a yeast, and the isolating the ethanol produced. Bush et al. further relates to processing of the biomass remaining after ethanol production to recovering biodiesel starting materials and/or generation of heat and carbon dioxide via combustion. Algal cells overexpressing the TpGPAT1 gene as described herein are used in the process of Bush et al. to produce ethanol and biodiesel.

For instance, as described in Bush et al., lipids/oils, which are useful for forming biodiesel typically, remain in the biomass after it has been subjected to fermentation, and the fermentation solution has been removed. These lipids/oils are isolated from the biomass and then used to form biodiesel using methods known to form biodiesel. A convenient method of separating lipids/oils from the biomass is by pressure. For example, the biomass can be pressed and the resulting lipid-rich liquid separated.

Thus, a process for forming biodiesel starting materials comprises recovering the lipids/oils remaining in the biomass after fermentation and ethanol separating. This process can further comprise: converting the recovered lipids/oils into biodiesel. For instance, U.S. Patent Application 20070048848 to Sears et al. (Mar. 1, 2007), the contents of the entirety of which are incorporated by this reference, describe a “Method, apparatus and system for biodiesel production from algae.” See, also, separating oil from the algal cells and processing it into diesel using standard transesterification technologies such as the Connemann process (see, e.g., U.S. Pat. No. 5,354,878, the entire contents of which are incorporated herein by this reference).

TABLE 1
Table 1. Fatty acid composition of the lipids from yeast gat1mutants transformed with the TpGPAT1
and pYES2.1 empty vector pYES2.1.

| | Proportion of Fatty Acid (mol %) | | | | | |
|---|---|---|---|---|---|---|
| Lipid fraction | 16:0 | 16:1 | 18:0 | 18:1 | Sats | Unsats |
| TAG | | | | | | |
| TpGPAT1 | 41.80 ± 2.17 | 29.15 ± 1.61 | 9.17 ± 1.22 | 18.94 ± 0.99 | 50.97 ± 2.85 | 48.49 ± 2.44 |
| pYES2.1 | 23.53 ± 4.33 | 42.58 ± 4.48 | 5.86 ± 0.78 | 26.91 ± 1.41 | 29.39 ± 4.94 | 69.93 ± 5.55 |
| Phospholipids | | | | | | |
| TpGPAT1 | 31.10 ± 3.78 | 34.15 ± 2.74 | 8.92 ± 1.49 | 25.45 ± 2.25 | 40.02 ± 4.57 | 59.83 ± 4.61 |
| pYES2.1 | 19.34 ± 2.06 | 41.69 ± 1.13 | 5.53 ± 0.79 | 33.19 ± 2.56 | 24.87 ± 1.30 | 74.97 ± 1.42 |

Yeast cells were harvested after 3-day induction with 2% galactose.
Values, expressed as mol % of total fatty acids represent the average ± SD for three replicates.

TABLE 2
Table 2. Incorporation of EPA or DHA into TAGs and phospholipids yeast gat1
mutants transformed with the TpGPAT1 and pYES2.1.

| | | | | mol % of FA in | |
|---|---|---|---|---|---|
| Feeding | | mol % of FA in TAGS | | Phospholipids | |
| concentration | Construct | EPA | DHA | EPA | DHA |
| 50 μM | TpGPAT1 | 1.18 | 0.70 ± 0.06 | 0.42 ± 0.01 | 0.22 ± 0.03 |
| | pYES2.1 | 1.51 | 0.65 ± 0.19 | 0.51 | 0.17 ± 0.01 |
| 100 μM | TpGPAT1 | 3.38 ± 0.78 | 1.10 ± 0.07 | 0.85 ± 0.15 | 0.34 ± 0.02 |
| | pYES2.1 | 3.60 ± 1.94 | 1.37 ± 0.05 | 1.12 ± 0.40 | 0.28 ± 0.04 |

Yeast cells were harvested after 3-day induction with 2% galactose in the presence of EPA or DHA.
Values, expressed as mol % of total fatty acids represent the average ± SD for three replicates.

### REFERENCES

### The Contents of Each of which are Incorporated Herein by this Reference

1. Guschina, L A, and Harwood, J. L. (2006). Lipids and lipid metabolism in eukaryotic algae. Prog Lipid Res 45, 160-186.
2. Brent, M. T., apd Miller-Navarra, D. (1997). The role of highly unsaturated fatty acids in aquatic foodweb processes. Freshwater Biology 38,483-499.
3. Tonon, T., Harvey, D., Larson, T. R., and Graham, L A. (2002). Long chain polyunsaturated fatty acid production and partitioning to triacylglycerols in four microalgae. Phytochemistry 61, 15-24.
4. Tonon T, Qing R, Harvey D, Li Y, Larson T. R, and Graham L A. (2005). Identification of a long-chain polyunsaturated fatty acid acyl-coenzyme A synthetase from the diatom T. pseudonana. Plant Physiol 138, 402-408.
5. Zheng Z., and Zou J. (2001). The initial step of the glycerolipid pathway: identification of glycerol 3-phosphate/dihydroxyacetone phosphate dual substrate acyltransferases in Saccharomyces cerevisiae. J Biol. Chem. 276(45):41710-41716.
6. Bradford MhrI (1976). A rapid and sensitive method for the quantification of microgram quantities of protein utilizing the principle of protein-dye binding. Anal Biochem 72: 248-254
7. Schneiter, R and Daum, G. (2005). Extraction of yeast lipids. Methods Mol. Biol., 313, 41-46.
8. Santiago T. C, Zufferey R, Mehra R. S, Coleman R. A, and Mamoun C. B. (2004). The Plasmodium falciparum PfGatp is an endoplasmic reticulum membrane protein important for the initial step of malarial glycerolipid synthesis. J Biol Chem 279:9222-9232.
9. Zufferey R, and Mamoun C. B. (2005). The initial step of glycerolipid metabolism in Leishmania major promastigotes involves a single glycerol-3-phosphate acyltransferase enzyme important for the synthesis of triacylglycerol but not essential for virulence. Mol. Microbial. 56:800-810.17
10. Lewin T M, Wang P, Coleman R A. (1999). Analysis of amino acid motifs diagnostic for the sn-glycerol-3-phosphate acyltransferase reaction. Biochemistry 38: 5764-5771
11. Zaremberg V, and McMaster C. R. (2002). Differential partitioning of lipids metabolized by separate yeast glycerol-3-phosphate acyltransferases reveals that phospholipase D generation of phosphatidic acid mediates sensitivity to choline-containing lysolipids and drugs. J Biol. Chem. 277:39035-39044.
12. Zheng Z, Xia Q, Dauk M, Shen W, Selvaraj G, and Zou J. (2003). Arabidopsis AtGPAT1, a member of the membrane-bound glycerol-3-phosphate acyltransferase gene family, is essential for tapetum differentiation and male fertility. Plant Cell 15: 1872-1887.
13. Cao, J., Lib J. L., Li, D., Tobin, J. F., Gimeno, R. E. (2006). Molecular identification of microsomal acyl-CoA:glycerol-3-phosphate acyltransferase, a key enzyme in de novo triacylglycerol synthesis. Proc Natl Acad Sci USA. 103:19695-19700.
14. Koncz, C., and Schell, J. (1986). The promoter of TL-DNA gene 5 controls the tissue-specific expression of chimaeric genes carried by a novel type of Agrobacterium vector. Mol. Gen. Genet. 204: 383-396.
15. Clough, S. J., and Bent, A. F. (1998). Floral dip: a simplified method for Agrobacterium-mediated transformation of Arabidopsis thaliana. Plant J. 16: 735-743.
16. Zheng Z., and Zou J. (2001) The initial step of the glycerolipid pathway: identification of glycerol 3-phosphate/dihydroxyacetone phosphate dual substrate acyltransferases in Saccharomyces cerevisiae. J Biol. Chem. 276(45): 41710-41716.
   These references are all hereby incorporated by this reference in their entirety.

What is claimed is:

1. An isolated, purified or recombinantly produced polypeptide comprising a sequence having at least 50% sequence identity to the sequence of SEQ ID NO:2, wherein the polypeptide further comprises:
a sequence having at least 90% identity to the sequence of SEQ ID NO:28;
a sequence having at least 90% identity to the sequence of SEQ ID NO:29;
a sequence having at least 90% identity to the sequence of SEQ ID NO:30; and
a sequence having at least 90% identity to the sequence of SEQ ID NO:31.

2. The isolated, purified or recombinantly produced polypeptide of claim 1, wherein the polypeptide consists of the amino acid sequence of SEQ ID NO:2.

3. The isolated, purified or recombinantly produced polypeptide of claim 1 having glycerol-3-acyltransferase activity.

4. An isolated or purified nucleic acid sequence encoding the isolated, purified or recombinantly produced polypeptide of claim 1.

5. The nucleic acid sequence of claim 4, wherein the nucleic acid sequence is present in a vector.

6. A transgenic plant including the isolated or purified nucleic acid sequence of claim 4.

7. A transgenic plant including the isolated or purified nucleic acid sequence of claim 4, wherein the plant has altered levels of palmitate oils as compared to levels in a plant lacking the nucleic acid sequence.

8. A yeast cell transformed with the isolated or purified nucleic acid sequence of claim 4.

9. An algal cell transformed with the isolated or purified nucleic acid sequence of claim 4.

10. A method of altering levels of high-palmitate oils in a plant, the method comprising:
introducing into the plant a nucleic acid comprising a polynucleotide encoding a polypeptide having at least 90% sequence identity to a polypeptide consisting of SEQ ID NO:2.

11. The method according to claim 10, wherein the nucleic acid construct is introduced into the plant by Agrobacterium-mediated transformation.

12. The method according to claim 10, the method further comprising:
introducing a polynucleotide encoding a polypeptide with Brassica pyruvate dehydrogenase kinase activity into the plant.

13. The method according to claim 10, the method further comprising:
introducing a polynucleotide encoding a polypeptide with diacylglycerol acetyltransferase activity.

14. The method according to claim 10, the method further comprising:
introducing a polynucleotide encoding a polypeptide with glycerol-3-phosphate dehydrogenase activity.

15. The method according to claim 10, wherein the plant is selected from the group consisting of Arabidopsis thaliana, Borago spp., Canola, Ricinus spp., Theobroma spp., Zea spp., Gossypium spp, Crambe spp., Cuphea spp., Linum spp., Lesquerella spp., Limnanthes spp., Linola, Tropaeolum spp., Oenothera spp., Olea spp., Elaeis spp., Arachis spp., rapeseed, Carthamus spp., Glycine spp., Soja spp., Helianthus spp., Nicotiana spp., Vernonia spp., Triticum spp., Hordeum spp., Oryza spp., Avena spp., Sorghum spp., Secale spp., Brassicaceae, and other members of the plant family Gramineae.

16. A plant produced by the method according to claim 10.

17. A seed harvested from the plant of claim 16.

18. Oil extracted from the plant of claim 16.

19. The method according to claim 10, further comprising:
harvesting a seed from the plant including the nucleic acid construct; and
extracting oil from the harvested seed.

20. In a process of producing biodiesel from algal cells, the improvement comprising:
using, as an algal cell in the process, the algal cell of claim 9.


SEQUENCE LISTING (31 sequences)

SEQ ID NO: 1 - DNA, 2025 bp, Thalassiosira pseudonana
atgggcgttgaaaagaagggcacaatgatgtccgagttggactatacgaaggcacaactcgccttcttctacatcgtcgtccttctatcactcgatatgctcaacccagtcaagatctttttacacgtctttcctgcaattaagtcatggcacatcgcgacatttgcaattgcctgcatgtcatacatcttcatcgtgaacttgagggaactgctatacttcgctaccaaggtcttcttccattcaatcctatcaatctttttcaacgacgtgaccgtggttggcagagagaatatcccgagccatggccctgttatctttacctccaaccacgctaatcagtttatggatgggttgatgattatgtgtacttgccaaaggacgatctcgtatcttgtagcagacaagtcttggaatagaccaatcattgggcatctggcttggatgatggggggagtgccagtcaaacgtgcacaagatagtgcctgtaaaggaactggaaaaatcagcattgacttgaacgctctcgcgggatcggatgcagtcatcaatgtcgttggaaagggaacatcgttcacgtctcagataaaagccggggataagattcgcctaccaaacaatgcaatcggcatcaaagttgaatctatcgaaagtgatgaatcaatgtcgctcaaagtggaagatggtgtggctgaagtattatcatcccatccatttcctgagtacgtcacatacgatattctgcctcgaattgatcagaaggacgtctaccaaaatgtactggagaaactagcatcaggcgggacgattggaatctttccagagggtggctcccacgataggactgacttgctcccattgaaagttggtgtggcactcattgcatactcggaacttgaaaaggatggaatcaacgtgccgattgtcccagttggattgaactactttcgggctcatcgcttccgtggcaaagcaactgttgagtttggttctccaacttatattgaaccatcgacacttgcagactacaaaaagggaggtgccgataagcgacgcgtttgcaatgatcttttggctcgtattgagaacagtatgagatctgtcattgtgtcggtgcccgactttgaaacacttcagaccatccatgctgcgaggaggctatacagacaagatggcagaaacgaaactgctgaacaaaggcaggacatgggcaggcgatttgcagaggggtacaaacgcgttcttctccaattaggaggagagccgccagaggagtggctcagtctgcagtcgaggatattggcgtaccaaaaggaattgaacgagctcggtatcagggactatcaggttgttggtcttgatcacgaggaggtggaactcggttcagagtcgcaaggtcattccaaagcagatactgttcttcacaggatgaatgtgtttggacacatcgttcatctgtttgtcattgcggtcttggcagcactgccagctatgttgttaaaccttccagttgggttggcatcacgaatctactccaatcgtaggcgaaaggttgcattggcagcatcaaaagtgaaggtgaagggatatgatgtgatgctctcagaacgtgtactggcatgtatcgttctcgttccttctctgtgggtagtgtatggattgcttctctccctgttcacctctctcgacggaccgtcacttgctgtatgcttcacctgctttcctttgttctcatattggagtattatggctacggaatcaggaatggttgatatcaaagatttgaggccgtacgttatgagaatgattccatcagcacgacgcaggttagcagcattgccagcaacgaggaaggctctccggtcggatcttagagcaatgattaagaagattggccccagcttgggtgatatttactacgaaaaggacttgaactggcagaagattcaaatggagacgaagaggatgtcaatggaggagttggatccagctcaaaaagacgaagcagcgaagaaggaagagtaa

SEQ ID NO: 2 - protein, 674 aa, Thalassiosira pseudonana
MGVEKKGTMMSELDYTKAQLAFFYIVVLLSLDMLNPVKIFLHVFPAIKSWHIATFAIACMSYIFIVNLRELLYFATKVFFHSILSIFFNDVTVVGRENIPSHGPVIFTSNHANQFMDGLMIMCTCQRTISYLVADKSWNRPIIGHLAWMMGGVPVKRAQDSACKGTGKISIDLNALAGSDAVINVVGKGTSFTSQIKAGDKIRLPNNAIGIKVESIESDESMSLKVEDGVAEVLSSHPFPEYVTYDILPRIDQKDVYQNVLEKLASGGTIGIFPEGGSHDRTDLLPLKVGVALIAYSELEKDGINVPIVPVGLNYFRAHRFRGKATVEFGSPTYIEPSTLADYKKGGADKRRVCNDLLARIENSMRSVIVSVPDFETLQTIHAARRLYRQDGRNETAEQRQDMGRRFAEGYKRVLLQLGGEPPEEWLSLQSRILAYQKELNELGIRDYQVVGLDHEEVELGSESQGHSKADTVLHRMNVFGHIVHLFVIAVLAALPAMLLNLPVGLASRIYSNRRRKVALAASKVKVKGYDVMLSERVLACIVLVPSLWVVYGLLLSLFTSLDGPSLAVCFTCFPLFSYWSIMATESGMVDIKDLRPYVMRMIPSARRRLAALPATRKALRSDLRAMIKKIGPSLGDIYYEKDLNWQKIQMETKRMSMEELDPAQKDEAAKKEE

SEQ ID NO: 3 - protein, 674 aa, Artificial Sequence
  Prophetic example
MGVEKKGTMMSELDYTKAQLAFFYIVVLLSLDMLNPVKIFLHVFPAIKSWHIATFAIACMSYIFIVNLRELLYFATKVFFHSILSIFFNDVTVVGRENIPSHGPVIFTSNHANQFMDGLMIMCTCQRTISYLVADKSWNRPIIGHLAWMMGGVPVKRAQDSACKGTGKISIDLNALAGSDAVINVVGKGTSFTSQIKAGDKIRLPNNAIGIKVESIESDESMSLKVEDGVAEVLSSHPFPEYVTYDILPRIDQKDVYQNVLEKLASGGTIGIFPEGGSHDRTDLLPLKVGVALIAYSELEKDGINVPIVPVGLNYFRAHRFRGKATVEFGSPTYIEPSTLADYKKGGADKRRVCNDLLARIENSMRSVIVSVPDFETLQTIHAARRLYRQDGRNETAEQRQDMGRRFAEGYKRVLLQLGGEPPEEWLSLQSRILAYQKELNELGIRDYQVVGLDHEEVELGSESQGHSKADTVLHRMNVFGHIVHLFVIAVLAALPAMLLNLPVGLASRIYSNRRRKVALAASKVKVKGYDVMLSERVLACIVLVPSLWVVYGLLLSLFTSLDGPSLAVCFTCFPLFSYWSIMATESGMVDIKDLRPYVMRMIPSARRYDVSSDATRKALRSDLRAMIKKIGPSLGDIYYEKDLNWQKIQMETKRMSMEELDPAQKDEAAKKEE

SEQ ID NO: 4 - protein, 674 aa, Artificial Sequence
  Prophetic example
MGVEKKGTMMSELDYTKAQLAFFYIVVLLSLDMLNPVKIFLHVFPAIKSWHIATFAIACMSYIFIVNLRELLYFATKVFFHSILSIFFNDVTVVGRENIPSHGPVIFTSNHANQFMDGLMIMCTCQRTISYLVADKSWNRPIIGHLAWMMGGVPVKRAQDSACKGTGKISIDLNALAGSDAVINVVGKGTSFTSQIKAGDKIRLPNNAIGIKVESIESDESMSLKVEDGVAEVLSSHPFPEYVTYDILPRIDQKDVYQNVLEKLASGGTIGIFPEGGSHDRTDLLPLKVGVALIAYSELEKDGINVPIVPVGLNYFRAHRFRGKATVEFGSPTYIEPSTLADYKKGGADKRRVCNDLLARIENSMRSVIVSVPDFETLQTIHAARRLYRQDGRNETAEQRQDMGRRFAEGYKRVLLQLGGEPPEEWLSLQSRILAYQKELNELGIRDYQVVGLDHEEVELGSESQGHSKADTVLHRMNVFGHIVHLFVIAVLAALPAMLLNLPVGLASRIYSNRRRKVALAASKVKVKGYDVMLSERVLACIVLVPSLWVVYGLLLSLFTSLDGPSLAVCFTCFPLFSYWSIMATESGMVDIKDLRPYVMRMIPSARRRKTSELNRRKALRSDLRAMIKKIGPSLGDIYYEKDLNWQKIQMETKRMSMEELDPAQKDEAAKKEE

SEQ ID NO: 5 - protein, 674 aa, Artificial Sequence
  Prophetic example
MGVEKKGTMMMELWPGAWTALLQLLLLLLSTLWFCSSSAKYFFKMAFYSWHIATFAIACMSYIFIVNLRELLYFATKVFFHSILSIFFNDVTVVGRENIPSHGPVIFTSNHANQFMDGLMIMCTCQRPVRFLMAEKSFQRPVIGHLAWMMGGVPVKRAQDSACKGTGKISIDLNALAGSDAVINVVGKGTSFTSQIKAGDKIRLPNNAISWGVPGNAKCRAQVSRITSDEEVELSSHPFPEYVTYDILPRIDQKDVYQNVLEKLASGGTIGIFPEGGSHDRTDLLPLKVGVALIAYSELEKDGINVPIVPVGLNYFRAHRFRGKATVEFGSPTYIEPSTLADYKKGGADKRRVCNDLLARIENSMRSVIVSVPDFETLQTIHAARRLYRQDGRNETAEQRQDMGRRFAEGYKRVLLQLGGEPPEEWLSLQSRILAYQKELNELGIRDYQVVGLDHEEVELGSESQGHSKADTVLHRMNVFGHIVHLFVIAVLAALPAMLLNLPVGLASRIYSNRRRKVALAASKVKVKGYDVMLSERVLACIVLVPSLWVVYGLLLSLFTSLDGPSLAVCFTCFPLFSYWSIMATESGMVDIKDLRPYVMRMIPSARRRLAALPATRKALRSDLRARSRSSGPSLGDIYYEKDLNWQKIQMETKRMSMEELDPAQKDEAAKKEE

SEQ ID NO: 6 - protein, 674 aa, Artificial Sequence
  Prophetic example
MGVEKKGTMMSELDYTKAQLAFMPAPKLTEKFASSKKSTQKHVFPAIKSWHIATFAIACMSYIFIVNLRELLYFATKVFFHSILSIFFNDVTVVGRENIPSHGPVIFTSNHANQFMDGLMIMCTCQRPVRFLMAEKSFQRPVIGHLAWMMGGVPVKRAQDSACKGTGKISIDLNALAGSDAVINVVGKGTSFTSQIKAGDKIRLPNNAISWGVPGNAKCRAQVSRITSDEEVELSSHPFPEYVTYDILPRIDQKDVYQNVLEKLASGGTIGIFPEGGSHDRTDLLPLKVGVALIAYSELEKDGINVPIVPVGLNYFRAHRFRGKATVEFGSPTYIEPSTLADYKKGGADKRRVCNDLLARIENSMRSVIVSVPDFETLQTIHAARRLYRQDGRNETAEQRQDMGRRFAEGYKRVLLQLGGEPPEEWLSLQSRILAYQKELNELGIRDYQVVGLDHEEVELGSESQGHSKADTVLHRMNVFGHIVHLFVIAVLAALPAMLLNLPVGLASRIYSNRRRKVALAASKVKVKGYDVMLSERVLACIVLVPSLWVVYGLLLSLFTSLDGPSLAVCFTCFPLFSYWSIMATESGMVDIKDLRPYVMRMIPSARRRLAALPATRKALRSDLRARSRSSGPSLGDIYYEKDLNWQKIQMETKRMSMEELDPAQKDEAAKKEE

SEQ ID NO: 7 - protein, 674 aa, Artificial Sequence
  Prophetic example
MGVEKKGTMMSELDYTKAQLAFFYIVVLLSLDMLNPVKIFLHVFPAIKSWHIATFAIACMSYIFIVNLRELLYFATKVFFHSILSIFFNDVTVVGRENIPSHGPVIFTSNHANQFMDGLMIMCTCQRTISYLVADKSWNRPIIGHLAWMMGGVPVKRAQDSACKGTGKISIDLNALAGSDAVINVVGKGTSFTSQIKAGDKIRLPNNAIGIKVESIESDESMSLKVEDGVAEVLSSHPFPEYVTYDILPRIDQKDVYQNVLEKLASGGTIGIFPEGGSHDRTDLLPLKVGVALIAYSELEKDGINVPIVPVGLNYFRAHRFRGKATVEFGSPTYIEPSTLADYKKGGADKRRVCNDLLARIENSMRSVIVSVPDFETLQTIHAARRLYRQDGRNETAEQRQDMGRRFAEGYKRVLLQLGGEPPEEWLSLQSRILAYQKELNELGIRDYQVVGLDHEEVELGSESQGHSKADTVLHRMNVFGHIVHLFVIAVLAALPAMLLNLPVGLASRIYSNRRRKVALAASKVKVKGYDVMLSERVLACIVLVPSLWVVYGLLLSLFTSLDGPSLAVCFTCFPLFSYWSIMATEIGMDGFKSLRPLVLSLTSPARRRLAALPATRKALRSDLRAMIKKIGPSLGDIYYEKDLNWQKIQMETKRMSMEELDPAQKDEAAKKEE

SEQ ID NO: 8 - DNA, 2025 bp, Artificial Sequence
  Prophetic example
atgggtgtcgagaaaaaaggaacgatgatgtccgagttggactatacgaaggcacaactcgccttcttctacatcgtcgtccttctatcactcgatatgctcaacccagtcaagatctttttacacgtctttcctgcaattaagtcatggcacatcgcgacatttgcaattgcctgcatgtcatacatcttcatcgtgaacttgagggaactgctatacttcgctaccaaggtcttcttccattcaatcctatcaatctttttcaacgacgtgaccgtggttggcagagagaatatcccgagccatggccctgttatctttacctccaaccacgctaatcagtttatggatgggttgatgattatgtgtacttgccaaaggacgatctcgtatcttgtagcagacaagtcttggaatagaccaatcattgggcatctggcttggatgatggggggagtgccagtcaaacgtgcacaagatagtgcctgtaaaggaactggaaaaatcagcattgacttgaacgctctcgcgggatcggatgcagtcatcaatgtcgttggaaagggaacatcgttcacgtctcagataaaagccggggataagattcgcctaccaaacaatgcaatcggcatcaaagttgaatctatcgaaagtgatgaatcaatgtcgctcaaagtggaagatggtgtggctgaagtattatcatcccatccatttcctgagtacgtcacatacgatattctgcctcgaattgatcagaaggacgtctaccaaaatgtactggagaaactagcatcaggcgggacgattggaatctttccagagggtggctcccacgataggactgacttgctcccattgaaagttggtgtggcactcattgcatactcggaacttgaaaaggatggaatcaacgtgccgattgtcccagttggattgaactactttcgggctcatcgcttccgtggcaaagcaactgttgagtttggttctccaacttatattgaaccatcgacacttgcagactacaaaaagggaggtgccgataagcgacgcgtttgcaatgatcttttggctcgtattgagaacagtatgagatctgtcattgtgtcggtgcccgactttgaaacacttcagaccatccatgctgcgaggaggctatacagacaagatggcagaaacgaaactgctgaacaaaggcaggacatgggcaggcgatttgcagaggggtacaaacgcgttcttctccaattaggaggagagccgccagaggagtggctcagtctgcagtcgaggatattggcgtaccaaaaggaattgaacgagctcggtatcagggactatcaggttgttggtcttgatcacgaggaggtggaactcggttcagagtcgcaaggtcattccaaagcagatactgttcttcacaggatgaatgtgtttggacacatcgttcatctgtttgtcattgcggtcttggcagcactgccagctatgttgttaaaccttccagttgggttggcatcacgaatctactccaatcgtaggcgaaaggttgcattggcagcatcaaaagtgaaggtgaagggatatgatgtgatgctctcagaacgtgtactggcatgtatcgttctcgttccttctctgtgggtagtgtatggattgcttctctccctgttcacctctctcgacggaccgtcacttgctgtatgcttcacctgctttcctttgttctcatattggagtattatggctacggaatcaggaatggttgatatcaaagatttgaggccgtacgttatgagaatgattccatcagcacgacgcaggttagcagcattgccagcaacgaggaaggctctccggtcggatcttagagcaatgattaagaagattggccccagcttgggtgatatttactacgaaaaggacttgaactggcagaagattcaaatggagacgaagaggatgtcaatggaggagttggatccagctcaaaaagacgaagcagcgaagaaggaagagtaa

SEQ ID NO: 9 - DNA, 2025 bp, Artificial Sequence
  Prophetic example
atgggtgtcgagaaaaaaggaacgatgatgtccgagttggactatacgaaggcacaactcgccttcttctacatcgtcgtccttctatcactcgatatgctcaacccagtcaagatctttttacacgtctttcctgcaattaagtcatggcacatcgcgacatttgcaattgcctgcatgtcatacatcttcatcgtgaacttgagggaactgctatacttcgctaccaaggtcttcttccattcaatcctatcaatctttttcaacgacgtgaccgtggttggcagagagaatatcccgagccatggccctgttatctttacctccaaccacgctaatcagtttatggatgggttgatgattatgtgtacttgccaaaggacgatctcgtatcttgtagcagacaagtcttggaatagaccaatcattgggcatctggcttggatgatggggggagtgccagtcaaacgtgcacaagatagtgcctgtaaaggaactggaaaaatcagcattgacttgaacgctctcgcgggatcggatgcagtcatcaatgtcgttggaaagggaacatcgttcacgtctcagataaaagccggggataagattcgcctaccaaacaatgcaatcggcatcaaagttgaatctatcgaaagtgatgaatcaatgtcgctcaaagtggaagatggtgtggctgaagtattatcatcccatccatttcctgagtacgtcacatacgatattctgcctcgaattgatcagaaggacgtctaccaaaatgtactggagaaactagcatcaggcgggacgattggaatctttccagagggtggctcccacgataggactgacttgctcccattgaaagttggtgtggcactcattgcatactcggaacttgaaaaggatggaatcaacgtgccgattgtcccagttggattgaactactttcgggctcatcgcttccgtggcaaagcaactgttgagtttggttctccaacttatattgaaccatcgacacttgcagactacaaaaagggaggtgccgataagcgacgcgtttgcaatgatcttttggctcgtattgagaacagtatgagatctgtcattgtgtcggtgcccgactttgaaacacttcagaccatccatgctgcgaggaggctatacagacaagatggcagaaacgaaactgctgaacaaaggcaggacatgggcaggcgatttgcagaggggtacaaacgcgttcttctccaattaggaggagagccgccagaggagtggctcagtctgcagtcgaggatattggcgtaccaaaaggaattgaacgagctcggtatcagggactatcaggttgttggtcttgatcacgaggaggtggaactcggttcagagtcgcaaggtcattccaaagcagatactgttcttcacaggatgaatgtgtttggacacatcgttcatctgtttgtcattgcggtcttggcagcactgccagctatgttgttaaaccttccagttgggttggcatcacgaatctactccaatcgtaggcgaaaggttgcattggcagcatcaaaagtgaaggtgaagggatatgatgtgatgctctcagaacgtgtactggcatgtatcgttctcgttccttctctgtgggtagtgtatggattgcttctctccctgttcacctctctcgacggaccgtcacttgctgtatgcttcacctgctttcctttgttctcatattggagtattatggctacggaatcaggaatggttgatatcaaagatttgaggccgtacgttatgagaatgattccatcagcacgacgcaggttagcagcattgccagcaacgaggaaggctctccggtcggatcttagagcaatgattaagaagattggccccagcttgggtgatatttactacgaaaaggacttgaactggcagaagattcaaatggagacgaagaggatgtcaatggaggagttggatccagctcaaaaagacgaagcagcgaagaaagaggaataa

SEQ ID NO: 10 - DNA, 2025 bp, Artificial Sequence
  Prophetic example
atgggcgttgaaaagaagggcacaatgatgtccgagttggactatacgaaggcacaactcgccttcttctacatcgtcgtccttctatcactcgatatgctcaacccagtcaagatctttttacacgtctttcctgcaattaagtcatggcacatcgcgacatttgcaattgcctgcatgtcatacatcttcatcgtgaacttgagggaactgctatacttcgctaccaaggtcttcttccattcaatcctatcaatctttttcaacgacgtgaccgtggttggcagagagaatatcccgagccatggccctgttatctttacctccaaccacgctaatcagtttatggatgggttgatgattatgtgtacttgccaaaggacgatctcgtatcttgtagcagacaagtcttggaatagaccaatcattgggcatctggcttggatgatggggggagtgccagtcaaacgtgcacaagatagtgcctgtaaaggaactggaaaaatcagcattgacttgaacgctctcgcgggatcggatgcagtcatcaatgtcgttggaaagggaacatcgttcacgtctcagataaaagccggggataagattcgcctaccaaacaatgcaatcggcatcaaagttgaatctatcgaaagtgatgaatcaatgtcgctcaaagtggaagatggtgtggctgaagtattatcatcccatccatttcctgagtacgtcacatacgatattctgcctcgaattgatcagaaggacgtctaccaaaatgtactggagaaactagcatcaggcgggacgattggaatctttccagagggtggctcccacgataggactgacttgctcccattgaaagttggtgtggcactcattgcatactcggaacttgaaaaggatggaatcaacgtgccgattgtcccagttggattgaactactttcgggctcatcgcttccgtggcaaagcaactgttgagtttggttctccaacttatattgaaccatcgacacttgcagactacaaaaagggaggtgccgataagcgacgcgtttgcaatgatcttttggctcgtattgagaacagtatgagatctgtcattgtgtcggtgcccgactttgaaacacttcagaccatccatgctgcgaggaggctatacagacaagatggcagaaacgaaactgctgaacaaaggcaggacatgggcaggcgatttgcagaggggtacaaacgcgttcttctccaattaggaggagagccgccagaggagtggctcagtctgcagtcgaggatattggcgtaccaaaaggaattgaacgagctcggtatcagggactatcaggttgttggtcttgatcacgaggaggtggaactcggttcagagtcgcaaggtcattccaaagcagatactgttcttcacaggatgaatgtgtttggacacatcgttcatctgtttgtcattgcggtcttggcagcactgccagctatgttgttaaaccttccagttgggttggcatcacgaatctactccaatcgtaggcgaaaggttgcattggcagcatcaaaagtgaaggtgaagggatatgatgtgatgctctcagaacgtgtactggcatgtatcgttctcgttccttctctgtgggtagtgtatggattgcttctctccctgttcacctctctcgacggaccgtcacttgctgtatgcttcacctgctttcctttgttctcatattggagtattatggctacggaatcaggaatggttgatatcaaagatttgaggccgtacgttatgagaatgattccatcagcacgacgctacgatgtatcatcggatgcaacgaggaaggctctccggtcggatcttagagcaatgattaagaagattggccccagcttgggtgatatttactacgaaaaggacttgaactggcagaagattcaaatggagacgaagaggatgtcaatggaggagttggatccagctcaaaaagacgaagcagcgaagaaggaagagtaa

SEQ ID NO: 11 - DNA, 2025 bp, Artificial Sequence
  Prophetic example
atgggcgttgaaaagaagggcacaatgatgtccgagttggactatacgaaggcacaactcgccttcttctacatcgtcgtccttctatcactcgatatgctcaacccagtcaagatctttttacacgtctttcctgcaattaagtcatggcacatcgcgacatttgcaattgcctgcatgtcatacatcttcatcgtgaacttgagggaactgctatacttcgctaccaaggtcttcttccattcaatcctatcaatctttttcaacgacgtgaccgtggttggcagagagaatatcccgagccatggccctgttatctttacctccaaccacgctaatcagtttatggatgggttgatgattatgtgtacttgccaaaggacgatctcgtatcttgtagcagacaagtcttggaatagaccaatcattgggcatctggcttggatgatggggggagtgccagtcaaacgtgcacaagatagtgcctgtaaaggaactggaaaaatcagcattgacttgaacgctctcgcgggatcggatgcagtcatcaatgtcgttggaaagggaacatcgttcacgtctcagataaaagccggggataagattcgcctaccaaacaatgcaatcggcatcaaagttgaatctatcgaaagtgatgaatcaatgtcgctcaaagtggaagatggtgtggctgaagtattatcatcccatccatttcctgagtacgtcacatacgatattctgcctcgaattgatcagaaggacgtctaccaaaatgtactggagaaactagcatcaggcgggacgattggaatctttccagagggtggctcccacgataggactgacttgctcccattgaaagttggtgtggcactcattgcatactcggaacttgaaaaggatggaatcaacgtgccgattgtcccagttggattgaactactttcgggctcatcgcttccgtggcaaagcaactgttgagtttggttctccaacttatattgaaccatcgacacttgcagactacaaaaagggaggtgccgataagcgacgcgtttgcaatgatcttttggctcgtattgagaacagtatgagatctgtcattgtgtcggtgcccgactttgaaacacttcagaccatccatgctgcgaggaggctatacagacaagatggcagaaacgaaactgctgaacaaaggcaggacatgggcaggcgatttgcagaggggtacaaacgcgttcttctccaattaggaggagagccgccagaggagtggctcagtctgcagtcgaggatattggcgtaccaaaaggaattgaacgagctcggtatcagggactatcaggttgttggtcttgatcacgaggaggtggaactcggttcagagtcgcaaggtcattccaaagcagatactgttcttcacaggatgaatgtgtttggacacatcgttcatctgtttgtcattgcggtcttggcagcactgccagctatgttgttaaaccttccagttgggttggcatcacgaatctactccaatcgtaggcgaaaggttgcattggcagcatcaaaagtgaaggtgaagggatatgatgtgatgctctcagaacgtgtactggcatgtatcgttctcgttccttctctgtgggtagtgtatggattgcttctctccctgttcacctctctcgacggaccgtcacttgctgtatgcttcacctgctttcctttgttctcatattggagtattatggctacggaatcaggaatggttgatatcaaagatttgaggccgtacgttatgagaatgattccatcagcacgacgccgtaaaacatcagagttaaacaggaggaaggctctccggtcggatcttagagcaatgattaagaagattggccccagcttgggtgatatttactacgaaaaggacttgaactggcagaagattcaaatggagacgaagaggatgtcaatggaggagttggatccagctcaaaaagacgaagcagcgaagaaggaagagtaa

SEQ ID NO: 12 - protein, 583 aa, Plasmodium falciparum
MPDFYFLIRWLCKVIVKSVFRDVNVINPENVPLYGSVIFVGNHNNQFIDACVLIANIPRQVKFIVAEKSMRRAVIGKLASVIGCISVKRPQDLKFKGIGHICWNEGDVKITGINTRFRLDVQIGDKLLIQNKMFPVVKIESETELLIQEVINIECEDKMNGVPFKIIPKINQTEVYNLVTNSLKNGDTIGIFPEGGSHDRTNLLPLKPGVAIMTLCALADGIEDVSIIPVGLSYSKLYQLQGCATLFYGNAIIISQDLCKEYNNNNREAISKLLSKIEEGMRSCMLTSKDHETSRCIELCVSLYTPERMTISKNKIYNNLQLFCKMFWKFGNSKVIENLSYELKCYEKLLQANKIKDDEVWMLKQSTSAATLKFIEHICTFIFCVIFGMTFSLLWLPLVLISIYLAERHRKAALRNSTIKIQGGDVVSSYKVLVLIVLLPTFNIVYGLLFSIYLYHSWLKRILFVFLSMCILPICYYINLNYAVQIPSLLRQMKILLKVICGKINVWRDNERELISTRHELQLKVRDLVSTLGPDVSDDFLEQLYRNIPKFVVDVDTKRLIRGKDEFLPILQRSQLEYKEEIL

SEQ ID NO: 13 - protein, 628 aa, Leishmania major
MAPTSALKPKDRIVASTRRSKFVLCEECHQNVPIEEWPDHRDRLRKVSLVEEVSRFHHTLVRLMGEFLMWVLRSVYFREVTVVGRENIPCTGAVVFYGNHQNQFIDALMMHSHCGRPVRFLMAEKSFQRPVIGTLGRIFNSVPVVRPQDVPLVDGEGKLVRTEGKFIVGAGTHFSRLSKGDVLSWGVPGNAKCRAQVSRITSDEEVEVTVPIPAEHEVHTPTSFKYSVRIDHSEMYAQVYDTLQKNHCIGIFPEGGSHDHTSLLPLKAGVALFSLGAAERHISVKIVPVGLTYLYGHKFRSRAYIEFGEAFSPPDDLVKLFDTDKRKATGLFLEQLNAALRAVTINVPDYKTLSFLHSFRQLYQPPNCMLSGHDYLRLIRRLGNVIEEQRKSAEFAEFREKVENYSDFCKALMIRDSQAATLKRLLNADNEALQVGLLLRRVFALYLMAVILVPFFVVGLPIGGIIKYLALKQTKRALSESSVKIVGADVTGSFKVLFAFAVVPGVFLLVTFIVFLYSDSRTALVILFSLPMAMYVSLLILQEAIMELRAALPLLMSLVSQHMQFKKLYERREDLARQARLIVHKYDPQLEEEIKVHGNAGDSNDDLNREPSLFSLRYNVRRRQATNS

SEQ ID NO: 14 - protein, 743 aa, Saccharomyces cerevisiae
MSAPAADHNAAKPIPHVPQASRRYKNSYNGFVYNIHTWLYDVSVFLFNILFTIFFREIKVRGAYNVPEVGVPTILVCAPHANQFIDPALVMSQTRLLKTSAGKSRSRMPCFVTAESSFKKRFISFFGHAMGGIPVPRIQDNLKPVDENLEIYAPDLKNHPEIIKGRSKNPQTTPVNFTKRFSAKSLLGLPDYLSNAQIKEIPDDETIILSSPFRTSKSKVVELLTNGTNFKYAEKIDNTETFQSVFDHLHTKGCVGIFPEGGSHDRPSLLPIKAGVAIMALGAVAADPTMKVAVVPCGLHYFHRNKFRSRAVLEYGEPIVVDGKYGEMYKDSPRETVSKLLKKITNSLFSVTENAPDYDTLMVIQAARRLYQPVKVRLPLPAIVEINRRLLFGYSKFKDDPRIIHLKKLVYDYNRKLDSVGLKDHQVMQLKTTKLEALRCFVTLIVRLIKFSVFAILSLPGSILFTPIFIICRVYSEKKAKEGLKKSLVKIKGTDLLATWKLIVALILAPILYVTYSILLIILARKQHYCRIWVPSNNAFIQFVYFYALLVFTTYSSLKTGEIGVDLFKSLRPLFVSIVYPGKKIEEIQTTRKNLSLELTAVCNDLGPLVFPDYDKLATEIFSKRDGYDVSSDAESSISRMSVQSRSRSSSIHSIGSLASNALSRVNSRGSLTDIPIFSDAKQGQWKSEGETSEDEDEFDEKNPAIVQTARSSDLNKENSRNTNISSKIASLVRQKREHEKKE

SEQ ID NO: 15 - protein, 750 aa, Saccharomyces cerevisiae
MPAPKLTEKSASSKSTQKTTNYSSIEAKSIYQEPSATKKILYSIATWLLYNIFHCFFREIRGRGSFKVPQQGPVIFVAAPHANQFVDPVILMGEVKKSVNRRVSFLIAESSLKQPPIGFLASFFMAIGVVRPQDNLKPAEGTIRVDPTDYKRVIGHDTHFLTDCMPKGLIGLPKSMGFGEIQSIESDTSLTLRKEFKMAKPEIKTALLTGTTYKYAAKVDQSCVYHRVFEHLAHNNCIGIFPEGGSHDRTNLLPLKAGVAIMALGCMDRHPDVNVKIVPCGMNYFHPHKFRSRAVVEFGDPIEIPKELVAKYHNSETNRDAVKELLDTISKGLQSVTVTCSDYETLMVVQTIRRLYMTQFSTKLPLPLIVEMNRRMVKGYEFYRNDPKIADLTKDIMAYNAALRHYNLPDHLVEEAKVNFAKNLGLVFFRSIGLCILFSLAMPGIIMFSPVFILAKRISQEKARTALSKSTVKIKANDVIATWKILIGMGFAPLLYIFWSVLITYYLRHKPWNKIYVFSGSYISCVIVTYSALIVGDIGMDGFKSLRPLVLSLTSPKGLQKLQKNRRNLAERIIEVVNNFGSELFPDFDSAALREEFDVIDEEEEDRKTSELNRRKMLRKQKIKRQEKDSSSPIISQRDNHDAYEHHNQDSDGVSLVNSDNSLSNIPLFSSTFHRKSESSLASTSVAPSSSSEFEVENEILEEKNGLASKIAQAVLNKRIGENTAREEEEEEEEEEEEEEEEEEGKEGDA

SEQ ID NO: 16 - protein, 828 aa, Homo sapiens
MDESALTLGTIDVSYLPHSSEYSVGRCKHTSEEWGECGFRPTIFRSATLKWKESLMSRKRPFVGRCCYSCTPQSWDKFFNPSIPSLGLRNVIYINETHTRHRGWLARRLSYVLFIQERDVHKGMFATNVTENVLNSSRVQEAIAEVAAELNPDGSAQQQSKAVNKVKKKAKRILQEMVATVSPAMIRLTGWVLLKLFNSFFWNIQIHKGQLEMVKAATETNLPLLFLPVHRSHIDYLLLTFILFCHNIKAPYIASGNNLNIPIFSTLIHKLGGFFIRRRLDETPDGRKDVLYRALLHGHIVELLRQQQFLEIFLEGTRSRSGKTSCARAGLLSVVVDTLSTNVIPDILIIPVGISYDRIIEGHYNGEQLGKPKKNESLWSVARGVIRMLRKNYGCVRVDFAQPFSLKEYLESQSQKPVSALLSLEQALLPAILPSRPSDAADEGRDTSINESRNATDESLRRRLIANLAEHILFTASKSCAIMSTHIVACLLLYRHRQGIDLSTLVEDFFVMKEEVLARDFDLGFSGNSEDVVMHAIQLLGNCVTITHTSRNDEFFITPSTTVPSVFELNFYSNGVLHVFIMEAIIACSLYAVLNKRGLGGPTSTPPNLISQEQLVRKAASLCYLLSNEGTISLPCQTFYQVCHETVGKFIQYGILTVAEHDDQEDISPSLAEQQWDKKLPEPLSWRSDEEDEDSDFGEEQRDCYLKVSQSKEHQQFITFLQRLLGPLLEAYSSAAIFVHNFSGPVPEPEYLQKLHKYLITRTERNVAVYAESATYCLVKNAVKMFKDIGVFKETKQKRVSVLELSSTFLPQCNRQKLLEYILSFVVL

SEQ ID NO: 17 - protein, 792 aa, Homo sapiens
MLEGRCQTQPRSSPSGREASLWSSGFGMKLEAVTPFLGKYRPFVGRCCQTCTPKSWESLFHRSITDLGFCNVILVKEENTRFRGWLVRRLCYFLWSLEQHIPPCQDVPQKIMESTGVQNLLSGRVPGGTGEGQVPDLVKKEVQRILGHIQAPPRPFLVRLFSWALLRFLNCLFLNVQLHKGQMKMVQKAAQAGLPLVLLSTHKTLLDGILLPFMLLSQGLGVLRVAWDSRACSPALRALLRKLGGLFLPPEASLSLDSSEGLLARAVVQAVIEQLLVSGQPLLIFLEEPPGALGPRLSALGQAWVGFVVQAVQVGIVPDALLVPVAVTYDLVQDAPCDIDHASAPLGLWTGALAVLRSLWSRWGCSHRICSRVHLAQPFSLQEYIVSARSCWGGRQTLEQLLQPIVLGQCTAVPDTEKEQEWTPITGPLLALKEEDQLLVRRLSCHVLSASVGSSAVMSTAIMATLLLFKHQKLLGEFSWLTEEILLRGFDVGFSGQLRSLLQHSLSLLRAHVALLRIRQGDLLVVPQPGPGLTHLAQLSAELLPVFLSEAVGACAVRGLLAGRVPPQGPWELQGILLLSQNELYRQILLLMHLLPQDLLLLKPCQSSYCYCQEVLDRLIQCGLLVAEETPGSRPACDTGRQRLSRKLLWKPSGDFTDSDSDDFGEADGRYFRLSQQSHCPDFFLFLCRLLSPLLKAFAQAAAFLRQGQLPDTELGYTEQLFQFLQATAQEEGIFECADPKLAISAVWTFRDLGVLQQTPSPAGPRLHLSPTFASLDNQEKLEQFIRQFICS

SEQ ID NO: 18 - protein, 434 aa, Homo sapiens
MEGAELAGKILSTWLTLVLGFILLPSVFGVSLGISEIYMKILVKTLEWATIRIEKGTPKESILKNSASVGIIQRDESPMEKGLSGLRGRDFELSDVFYFSKKGLEAIVEDEVTQRFSSEELVSWNLLTRTNVNFQYISLRLTMVWVLGVIVRYCVLLPLRVTLAFIGISLLVIGTTLVGQLPDSSLKNWLSELVHLTCCRICVRALSGTIHYHNKQYRPQKGGICVANHTSPIDVLILTTDGCYAMVGQVHGGLMGIIQRAMVKACPHVWFERSEMKDRHLVTKRLKEHIADKKKLPILIFPEGTCINNTSVMMFKKGSFEIGGTIHPVAIKYNPQFGDAFWNSSKYNMVSYLLRMMTSWAIVCDVWYMPPMTREEGEDAVQFANRVKSAIAIQGGLTELPWDGGLKRAKVKDIFKEEQQKNYSKMIVGNGSLS

SEQ ID NO: 19 - protein, 827 aa, Mus musculus
MEESSVTVGTIDVSYLPSSSEYSLGRCKHTSEDWVDCGFKPTFFRSATLKWKESLMSRKRPFVGRCCYSCTPQSWERFFNPSIPSLGLRNVIYINETHTRHRGWLARRLSYILFVQERDVHKGMFATSVTENVLSSSRVQEAIAEVAAELNPDGSAQQQSKAIQKVKRKARKILQEMVATVSPGMIRLTGWVLLKLFNSFFWNIQIHKGQLEMVKAATETNLPLLFLPVHRSHIDYLLLTFILFCHNIKAPYIASGNNLNIPVFSTLIHKLGGFFIRRRLDETPDGRKDILYRALLHGHVVELLRQQQFLEIFLEGTRSRSGKTSCARAGLLSVVVDTLSSNTIPDILVIPVGISYDRIIEGHYNGEQLGKPKKNESLWSVARGVIRMLRKNYGYVRVDFAQPFSLKEYLEGQSQKPVSAPLSLEQALLPAILPSRPNDVADEHQDLSSNESRNPADEAFRRRLIANLAEHILFTASKSCAIMSTHIVACLLLYRHRQGIHLSTLVEDFFVMKEEVLARDFDLGFSGNSEDVVMHAIQLLGNCVTITHTSRKDEFFITPSTTVPSVFELNFYSNGVLHVFIMEAIIACSIYAVLNKRCSGGSAGGLGNLISQEQLVRKAASLCYLLSNEGTISLPCQTFYQVCHETVGKFIQYGILTVAEQDDQEDVSPGLAEQQWDKKLPELNWRSDEEDEDSDFGEEQRDCYLKVSQSKEHQQFITFLQRLLGPLLEAYSSAAIFVHNFSGPVPESEYLQKLHRYLITRTERNVAVYAESATYCLVKNAVKMFKDIGVFKETKQKRVSVLELSSTFLPQCNRQKLLEYILSFVVL

SEQ ID NO: 20 - protein, 801 aa, Mus musculus
MDTMLKSNPQTQQRSNHNGQETSLWSSSFGMKMEAITPFLGKYRPFMGRCCQTCTPKSWESLFHRSIMDLGFCNVILVKEENTRFRGWLVRRLCYFLWSLEQHIPTSFDASQKIMENTGVQNLLSGRVPGAAGEGQAPELVKKEVQRILGHIQTTPRPFLLRLFSWALLWFLNRLFLNVQLHKGQMKMVQKAVQEGSPLVFLSTHKSLLDGFLLPFVLFSQGLGVVRVALDSRTCSPALRALLRKLGGLFLPPEVNLSLDNSEGILARAVVRATVEELLTSGQPLLIFLEEPPGSPGPRLSALGQAWLGVVIQAVQAGIISDATLVPVAIAYDLVPDAPCNMNHDLAPLGLWTGALAVFRRLCNCWGCNRRVCVRVHLAQPFSLQEYTINARSCWDSRQTLEHLLQPIVLGECSVVPDTEKEQEWTPPTGLLLALKEEDQLLVRRLSRHVLSASVASSAVMSTAIMATLLLLKHQKGVVLSQLLGEFSWLTEETLLRGFDVGFSGQLRCLAQHTLSLLRAHVVLLRVHQGDLVVVPRPGPGLTHLARLSMELLPTFLSEAVGACAVRGLLAGRVPPEGPWELQGIELLSQNELYRQILLLLHLLPQDLLLPQPCQSSYCYCQEVLDRLIQCGLLVAEETPGSRPACDTGRQHLSAKLLWKPSGDFTDSESDDFEEPGGRCFRLSQQSRCPDFFLFLCRLLSPILKAFAQAATFLHLGQLPDSEVAYSEKLFQFLQACAQEEGIFECADPNLAISAVWTFKDLGVLQEMPSPTGPQLHLSPTFATRDNQDKLEQFIRQFICS

SEQ ID NO: 21 - protein, 438 aa, Mus musculus
MEGADLAVKLLSTWLTLVGGLILLPSAFGLSLGISEIYMKILVKTLEWATLRIQKGAPKESALKNSASVGIIQRDESPMEKGLSGLRGRDFELSDVFYFSKKGLEAIVEDEVTQRFSSEELVSWNLLTRTNVNFQYISPRLTMVWVLGVLVRYCFLLPLRVTLAFIGISLLIIGTTLVGQLPDSSLKNWLSELVHLTCCRICVRSLSGTIHYHNKQYRPQKGGICVANHTSPIDVLILATDGCYAMVGQVHGGLMGIIQRAMVKACPHVWFERSEIKDRHLVTKRLKEHIADKKKLPILIFPEGTCINNTSVMMFKKGSFEIGGTIYPVAIKYNPQFGDAFWNSSKYNLVSYLLRIMTSWAIVCDVWYMPPMTREEGEDAVQFANRVKSAIAVQGGLTELPWDGGLKRAKVKDTFKEEQQKNYSKMIVGNGSPNLARD

SEQ ID NO: 22 - protein, 807 aa, Escherichia coli
MSGWPRIYYKLLNLPLSILVKSKSIPADPAPELGLDTSRPIMYVLPYNSKADLLTLRAQCLAHDLPDPLEPLEIDGTLLPRYVFIHGGPRVFTYYTPKEESIKLFHDYLDLHRSNPNLDVQMVPVSVMFGRAPGREKGEVNPPLRMLNGVQKFFAVLWLGRDSFVRFSPSVSLRRMADEHGTDKTIAQKLARVARMHFARQRLAAVGPRLPARQDLFNKLLASRAIAKAVEDEARSKKISHEKAQQNAIALMEEIAANFSYEMIRLTDRILGFTWNRLYQGINVHNAERVRQLAHDGHELVYVPCHRSHMDYLLLSYVLYHQGLVPPHIAAGINLNFWPAGPIFRRLGAFFIRRTFKGNKLYSTVFREYLGELFSRGYSVEYFVEGGRSRTGRLLDPKTGTLSMTIQAMLRGGTRPITLIPIYIGYEHVMEVGTYAKELRGATKEKESLPQMLRGLSKLRNLGQGYVNFGEPMPLMTYLNQHVPDWRESIDPIEAVRPAWLTPTVNNIAADLMVRINNAGAANAMNLCCTALLASRQRSLTREQLTEQLNCYLDLMRNVPYSTDSTVPSASASELIDHALQMNKFEVEKDTIGDIIILPREQAVLMTYYRNNIAHMLVLPSLMAAIVTQHRHISRDVLMEHVNVLYPMLKAELFLRWDRDELPDVIDALANEMQRQGLITLQDDELHINPAHSRTLQLLAAGARETLQRYAITFWLLSANPSINRGTLEKESRTVAQRLSVLHGINAPEFFDKAVFSSLVLTLRDEGYISDSGDAEPAETMKVYQLLAELITSDVRLTIESATQGEG

SEQ ID NO: 23 - protein, 585 aa, Arabidopsis thaliana
MVLPELLVILAEWVLYRLLAKSCYRAARKLRGYGFQLKNLLSLSKTQSLHNNSQHHLHNHHQQNHPNQTLQDSLDPLFPSLTKYQELLLDKNRACSVSSDHYRDTFFCDIDGVLLRQHSSKHFHTFFPYFMLVAFEGGSIIRAILLLLSCSFLWTLQQETKLRVLSFITFSGLRVKDMDNVSRSVLPKFFLENLNIQVYDIWARTEYSKVVFTSLPQVLVERFLREHLNADDVIGTKLQEIKVMGRKFYTGLASGSGFVLKHKSAEDYFFDSKKKPALGIGSSSSPQDHIFISICKEAYFWNEEESMSKNNALPRERYPKPLIFHDGRLAFLPTPLATLAMFIWLPIGFLLAVFRISVGVFLPYHVANFLASMSGVRITFKTHNLNNGRPEKGNSGVLYVCNHRTLLDPVFLTTSLGKPLTAVTYSLSKFSEFIAPLKTVSLKRDRKKDGEAMQRLLSKGDLVVCPEGTTCREPYLLRFSPLFAELTEDIVPVAVDARVSMFYGTTASGLKCLDPIFFLMNPRPVYCLEILKKLPKEMTCAGGKSSFEVANFIQGELARVLGFECTNLTRRDKYLVLAGNEGIVR

SEQ ID NO: 24 - protein, 501 aa, Arabidopsis thaliana [sequence omitted]

SEQ ID NO: 25 - protein, 680 aa, Homo sapiens
MESSSSSNSHFSVGPTSPSAVVLLYSKELKKWDEFEDILEERRHVSDLKFAMKCYTPLVYKGITPCKPIDIKCSVLNSEEIHYVIKQLSKESLQSVDVLREEVSEILDEMSHKLRLGAIRFCAFTLSKVFKQIFSKVCVNEEGIQKLQRAIQEHPVVLLPSHRSYIDFLMLSFLLYNYDLPVPVIAAGMDFLGMKMVGELLRMSGAFFMRRTFGGNKLYWAVFSEYVKTMLRNGYAPVEFFLEGTRSRSAKTLTPKFGLLNIVMEPFFKREVFDTYLVPISISYDKILEETLYVYELLGVPKPKESTTGLLKARKILSENFGSIHVYFGDPVSLRSLAAGRMSRSSYNLVPRYIPQKQSEDMHAFVTEVAYKMELLQIENMVLSPWTLIVAVLLQNRPSMDFDALVEKTLWLKGLTQAFGGFLIWPDNKPAEEVVPASILLHSNIASLVKDQVILKVDSGDSEVVDGLMLQHITLLMCSAYRNQLLNIFVRPSLVAVALQMTPGFRKEDVYSCFRFLRDVFADEFIFLPGNTLKDFEEGCYLLCKSEAIQVTTKDILVTEKGNTVLEFLVGLFKPFVESYQIICKYLLSEEEDHFSEEQYLAAVRKFTSQLLDQGTSQCYDVLSSDVQKNALAACVRLGVVEKKKINNNCIFNVNEPATTKLEEMLGCKTPIGKPATAKL

SEQ ID NO: 26 - protein, 678 aa, Mus musculus
MDVPSSSSSRFSVGSASPSSVLLYAKDLKKWDEFEDLLEERRHISDFKFAMKCYTPPLYRGITPCKPGDIKSIVLSSEEINYVIKQLSRESLTGVDVLREEASEILEEMSHKLRIGAIRFFAFVLSKIFKQIFSKVCVNEEGIQKLQRAVQEHPVVLLPSHRSYIDFLMLSFILYSYDLPVPVIAAGMDFLGMRVVSELLRMSGAFFMRRTFGGNKLYWAVFSEYVKTMLRCGYAPVEFFLEGTRSRAAKTLTPKFGLLNIVMEPFFKREVFDTYFVPISISYDKILEESLYAYEILGVPKPKESTTGLLKARRILSENFGSIHVYFGDPVSLRSLAAGRLNRNTYNLVPRCIPQKQPEDVQAFVTEVAYKMQLLQIENLALSPWLLVVTILLQNQLSMDFDALVEKTLWLKGVTQVFGGFLLWPDNKLPEEVVQSSILLHSNLASLVKDQVVLKMNSGSSQVVNGLVPEHIALLMCSAYRNQLLNIFARPSLVALALHMTPGLRKEDVFSCFSFLRNVFSDEFIFLPGNTLRDFEEGCYLLCKAEAMQMAGKDIILTDKGTAVLQFLTSLFKPFVESYQLLCRYLLHEEDYFGEKEYLVAARKFTRQLLDQGSSQCYDALSSELQKNALAAFVRLGVVEKKKVDSKYVYYVNGPATSKLEEMLGCKKPIGKPATAKL

SEQ ID NO: 27 - protein, 1436 aa, Leishmania major
MSFPPPRLALPEECKQVVLVAPIDGFTAACACAIATSPLVPLNCRVLVDGIAVKSSSPSSGNDSPHMACSSAESAATARDFMNTVQRVEAKLRASRATRRDVSVTHITQFYYGGVSPKAEEIGPMLHRIHLLGSGALGETNFTASSTDVTAGSGGSSGVPRVADSVFVLVLSAQSLSPDTESGLKAYDILLRRLWSAAGPSQAAETASQQTRVRGSLVILADDVYRTAACAMLSSIAKEVATAVDWVVLLAYAYAMDPPALEFGATEVAVQAASGISLGGTLPVLCAANALGVLRHFPVSRGEPATITPVDVALNAALLGYVWLCHGELPECVECAAEMQYCGLSPAPSHAAAAAETATAMPTQPAQCVTRHRMGSFAVAESRKPGEVSLVWGMVGEYLMSYYGRFADQISAAFPVAGVLTQAPILQFPFSVADWVNFGPEPRAAAYALEGWRAYQQRQRLMEEKVGNAEAAQALKAYVAQMDSVVRYIERTARAAAARDKISAAFAASRRRTASSSFSVPPYRLDDFNVALYHSLLRRLTSHHTLMPYHQCVALVEVDWEAYVSVIARAVLEHLARCVLRSSSGLQHVRSRFHALPVADSANIGSVVRSPPVVSAPDELAPASAAVPSSTKPEAAASAAEAPFAFPEPRRYFTNDFVYRGAERIPPFPSLLHKYFDCFVFLHRVGMTANGWRGDMTPGMTPQVLTSIIAQPNIQRLMTALAAKDGASKKDVEARAKRLLLECGDALNHVHCRTLGLMIRTIFHSIYGRVDVNNGSYERLHRYFAMPRVAVVFAPLHRSYIDFLILSEVLALMRLPLPHIVAGENFLSMGVLATLMRGSGALFMRRSFRDDPLYAALLKEYVRHLVRARRPLEFFIEGARSRTGKTMAPKLGLLKFVCDTFYEPGQQELDDVLIVPVSLSYDEVLEATTHAKELLGVPKPKENPTNMLKARSLLERMHGNIHIHVGEPVSLRSLKQHPRQCPLPLQPKDEQLRDPAAPAAATTSVCKTLDTTPSIAKGSSITPIPLLGTVAWHLVYKLQRNTVITPASMVAAVVECLGPYYCTATTMLKEGIDTTAAAPTAAAAMPLEKVHQGVEWLRGRLRERGAQLSAEASKLNPAGVVTLALTNLYRYIHITDEMSGVTYHPDNVVTRLAVNISTNQLIHVCMNEALVAVVAQAFGFVTTPHRPPSGADGAMLCGVRSVKADVLSNQTQLLQRLLSAEFPNYAEAAPVSFASWLECTVSQLQQCVEEHTVSSQLSGADTHGGTIVHLPVTQYYYFLLQLICPHVEALYTVLAAASALLAAYPGKPFGAAEVVTATQRACYILYAERKLRYIVAANKETLQHYYESLIALSLLQIKRVPITTTPGQSKVNGGVVAYTVGLLGQEAALARLEVLSTQIKALWWHPSAAEGVTIDKAAIQERVLAVYRELTQPSKM

SEQ ID NO: 28 - protein, 11 aa, Thalassiosira pseudonana
HANQFMDGLMI

SEQ ID NO: 29 - protein, 8 aa, Thalassiosira pseudonana
VPVKRAQD

SEQ ID NO: 30 - protein, 11 aa, Thalassiosira pseudonana
IGIFPEGGSHD

SEQ ID NO: 31 - protein, 8 aa, Thalassiosira pseudonana
IVPVGLNY